(19) United States
(12) Patent Application Publication
(10) Pub. No.: US 2009/0286834 A1
(43) Pub. Date: Nov. 19, 2009

(54) Pyridine Analogues VI

(75) Inventors: Fabrizio Giordanetto, Molndal (SE); Johan Johansson, Molndal (SE)

Correspondence Address: Pepper Hamilton LLP, 400 Berwyn Park, 899 Cassatt Road, Berwyn, PA, 19312-1183, US

(73) Assignee: AstraZeneca AB, Sodertalje (SE)

(21) Appl. No.: 12/307,281

(22) PCT Filed: Jul. 2, 2007
(86) PCT No.: PCT/SE07/00643

§ 371 Date: Jan. 2, 2009

(30) Foreign Application Priority Data
Jul. 4, 2006 (SE) 0601464-1

(51) Int. Cl. A61K 31/4439; C07D 413/02; C07D 211/72; A61K 31/44

(52) U.S. Cl. 514/340; 546/271.4; 546/312; 514/352

(57) ABSTRACT

The present invention relates to certain new pyridin analogues of Formula (I) to processes for preparing such compounds, to their utility as P2Y₁₂ inhibitors and as anti-trombotic agents etc, their use as medicaments in cardiovascular diseases as well as pharmaceutical compositions containing them.

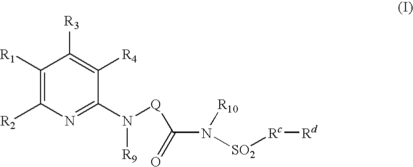

### FIELD OF THE INVENTION

The present invention provides novel pyridine compounds, their use as medicaments, compositions containing them and processes for their preparation.

### BACKGROUND OF THE INVENTION

Platelet adhesion and aggregation are initiating events in arterial thrombosis. Although the process of platelet adhesion to the sub-endothelial surface may have an important role to play in the repair of damaged vessel walls, the platelet aggregation that this initiates can precipitate acute thrombotic occlusion of vital vascular beds, leading to events with high morbidity such as myocardial infarction and unstable angina. The success of interventions used to prevent or alleviate these conditions, such as thrombolysis and angioplasty is also compromised by platelet mediated occlusion or re-occlusion.

Haemostasis is controlled via a tight balance between platelet aggregation, coagulation and fibrinolysis. Thrombus formation under pathological conditions, like e.g. arteriosclerotic plaque rupture, is firstly initiated by platelet adhesion, activation and aggregation. This results not only in the formation of a platelet plug but also in the exposure of negatively charged phospholipids on the outer platelet membrane promoting blood coagulation. Inhibition of the build-up of the initial platelet plug would be expected to reduce thrombus formation and reduce the number of cardiovascular events as was demonstrated by the antithrombotic effect of e.g. Aspirin (BMJ 1994; 308: 81-106 Antiplatelet Trialists'Collaboration. Collaborative overview of randomised trials of antiplatelet therapy, I: Prevention of death, myocardial infarction, and stroke by prolonged antiplatelet therapy in various categories of patients).

Platelet activation/aggregation can be induced by a variety of different agonists. However, distinct intracellular signalling pathways have to be activated to obtain fall platelet aggregation, mediated via G-proteins G_{q}, G_{12/13} and G₁ (Platelets, A D Michelson ed., Elsevier Science 2002, ISBN 0-12-493951-1; 197-213: D Woulfe, et al. Signal transduction during the initiation, extension, and perpetuation of platelet plug formation) In platelets, the G-protein coupled receptor P2Y₁₂ (previously also known as the platelet P_{2T}, P2T_{ac}, or P2_{cyc} receptor) signals via Gi, resulting in a lowering of intra-cellular cAMP and full aggregation (Nature 2001; 409: 202-207 G Hollopeter, et al. Identification of the platelet ADP receptor targeted by antithrombotic drugs.). Released ADP from dense-granules will positively feedback on the P2Y12 receptor to allow full aggregation.

Clinical evidence for the key-role of the ADP-P2Y₁₂ feedback mechanism is provided by the clinical use of clopidogrel, an thienopyridine prodrug which active metabolite selectively and irreversibly binds to the P2Y₁₂ receptor, that has shown in several clinical trials to be effective in reducing the risk for cardiovascular events in patients at risk (Lancet 1996; 348: 1329-39: CAPRIE Steering committee, A randomised, blinded, trial of clopidogrel versus aspirin in patients at risk of ischaemic events (CAPRIE); N Engl J Med 2001; 345 (7): 494-502): The Clopidogrel in Unstable Angina to prevent Recurrent Events Trial Investigators. Effects of clopidogrel in addition to aspirin in patients with acute coronary syndromes without ST-segment elevation.). In these studies, the clinical benefit of Clopidogrel treatment is associated with an increased rate of clinical bleeding. Published data suggest that reversible P2Y₁₂ antagonists could offer the possibility for high clinical benefit with a reduced bleeding risk as compared to thienopyridines (Sem Thromb Haemostas 2005; 31 (2): 195-204, van Giezen & RG Humphries. Preclinical and clinical studies with selective reversible direct P2Y₁₂ antagonists.

Accordingly it is an object of the present invention to provide potent, reversible and selective P2Y₁₂-antagonists as anti-trombotic agents.

### SUMMARY OF THE INVENTION

We have now surprisingly found that certain pyridine compounds of Formula (I) or a pharmaceutically acceptable salt thereof are reversible and selective P2Y₁₂ antagonists, hereinafter referred to as the compounds of the invention. The compounds of the invention unexpectedly exhibit beneficial properties that render them particularly suitable for use in the treatment of diseases/conditions as described below (See p. 49-50). Examples of such beneficial properties are high potency, high selectivity, and an advantageous therapeutic window.

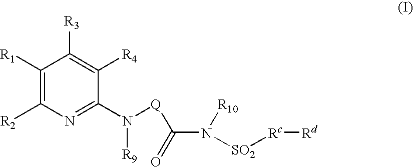

### DETAILED DESCRIPTION OF THE INVENTION

According to the present invention there is provided a novel compound of formula (I) or a pharmaceutically acceptable salt thereof:

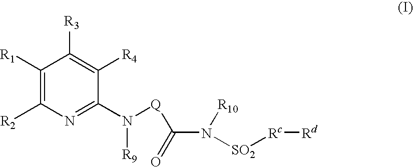

wherein
R₁ represents R₆OC(O), R₇C(O), R₁₆SC(O), R₁₇S, R₁₈C(S) or a group gII

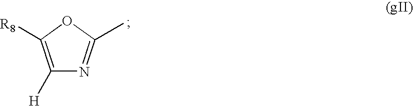

preferably R₁ represents R₆OC(O), R₁₆SC(O) or the group gII,

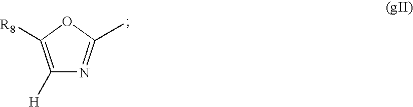

R₂ represents H, CN, halogen (F, Cl, Br, I), NO₂, (C₁-C₁₂)alkyl optionally interrupted by oxygen and/or optionally substituted by OH, aryl, cycloalkyl, heterocyclyl or one or more halogen (F, Cl, Br, I) atoms; further R₂ represents (C₁-C₁₂)alkoxy optionally substituted by one or more halogen (F, Cl, Br, I) atoms; further R₂ represents (C₃-C₆)cycloalkyl, hydroxy(C₁-C₂)alkyl, (C₁-C₁₂)alkylC(O), (C₁-C₁₂)alkylthioC(O), (C₁-C₁₂)alkylC(S), (C₁-C₁₂)alkoxyC(O), (C₃-C₆)cycloalkoxy, aryl, arylC(O), aryl(C₁-C₁₂)alkylC(O), heterocyclyl, heterocyclylC(O), heterocyclyl(C₁-C₁₂)alkylC(O), (C₁-C₁₂)alkylsulfinyl, (C₁-C₁₂)alkylsulfonyl, (C₁-C₁₂)alkylthio, (C₃-C₆)cycloalkylthio, arylsulfonyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl, (C₃-C₆)cycloalkyl(C₁-C₁₂)alkylsulfonyl or a group of formula NR^{a(2)}R^{b(2)} in which R^{b(2)} and R^{b(2)} independently represent H, (C₁-C₁₂)alkyl, (C₁-C₁₂)alkylC(O) or R^{a(2)} and R^{b(2)} together with the nitrogen atom represent piperidine, pyrrolidine, azetidine or aziridine;

R₃ represents H, CN, NO₂, halogen (F, Cl, Br, I), (C₁-C₁₂)alkyl optionally interrupted by oxygen and/or optionally substituted by OH, aryl, cycloalkyl, heterocyclyl or one or more halogen (F, Cl, Br, I) atoms; further R₃ represents (C₁-C₁₂)alkoxy optionally substituted by one or more halogen (F, Cl, Br, I) atoms; further R₃ represents (C₃-C₆)cycloalkyl, hydroxy(C₁-C₁₂)alkyl, (C₁-C₁₂)alkylC(O), (C₁-C₁₂)alkylthioC(O), (C₁-C₁₂)alkylC(S), (C₁-C₁₂)alkoxyC(O), (C₃-C₆)cycloalkoxy, aryl, arylC(O), aryl(C₁-C₁₂)alkylC(O), heterocyclyl, heterocyclylC(O), heterocyclyl(C₁-C₁₂)alkylC(O), (C₁-C₁₂)alkylsulfinyl, (C₁-C₁₂)alkylsulfonyl, (C₁-C₁₂)alkylthio, (C₃-C₆)cycloalkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl, (C₃-C₆)cycloalkyl(C₁-C₁₂)alkylsulfonyl or a group of formula NR^{a(3)}R^{b(3)} in which R^{a(3)} and R^{b(3)} independently represent H, (C₁-C₁₂)alkyl, (C₁-C₁₂)alkylC(O) or R^{a(3)} and R^{b(3)} together with the nitrogen atom represent piperidine, pyrrolidine, azetidine or aziridine;

R₄ represents H, CN, NO₂, halogen (F, Cl, Br, I), (C₁-C₁₂)alkyl optionally interrupted by oxygen and/or optionally substituted by OH, COOH, (C₁-C₆)alkoxycarbonyl, aryl, cycloalkyl, heterocyclyl or one or more halogen (F, Cl, Br, I) atoms; further R₄ represents (C₃-C₆)cycloalkyl, hydroxy(C₁-C₁₂)alkyl, (C₁-C₁₂)alkylC(O), (C₁-C₂)alkylcycloalkyl, (C₁-C₁₂)alkoxy wherein the alkoxy group may optionally be substituted by one or more halogen (F, Cl, Br, I) atoms, OH and/or COOH and/or (C₁-C₆)alkoxycarbonyl; further R₄ represents (C₁-C₁₂)alkylthioC(O), (C₁-C₁₂)alkylC(S), (C₁-C₁₂)alkoxyC(O), (C₃-C₆)cycloalkoxy, aryl, arylC(O), aryl(C₁-C₁₂)alkylC(O), heterocyclyl, heterocyclylC(O), heterocyclyl(C₁-C₂)alkylC(O), (C₁-C₁₂)alkylsulfinyl, (C₁-C₁₂)alkylsulfonyl, (C₁-C₁₂)alkylthio, (C₃-C₆)cycloalkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfonyl, (C₃-C₆)cycloalkyl(C₁-C₁₂)alkylsulfonyl or a group of formula NR^{a(4)}R^{b(4)} in which R^{b(4)} and R^{b(4)} independently represent H, (C₁-C₁₂)alkyl, (C₁-C₁₂)alkylC(O) or R^{a(4)} and R^{b(4)} together with the nitrogen atom represent piperidine, pyrrolidine, azetidine or aziridine;

R₆ represents (C₁-C₁₂)alkyl optionally interrupted by oxygen, (with the proviso that any such oxygen must be at least 2 carbon atoms away from the ester-oxygen connecting the R₆ group) and/or optionally substituted by OH, aryl, cycloalkyl, heterocyclyl or one or more halogen (F, Cl, Br, I) atoms; further R₆ represents (C₃-C₆)cycloalkyl, hydroxy(C₂-C₁₂)alkyl, aryl or heterocyclyl;

R₇ represents (C₁-C₁₂)alkyl optionally interrupted by oxygen, and/or optionally substituted by OH, aryl, cycloalkyl, heterocyclyl or one or more halogen (F, Cl, Br, I) atoms; further R₇ represents (C₃-C₆)cycloalkyl, hydroxy(C₁-C₁₂)alkyl, aryl or heterocyclyl;

R₈ represents H, (C₁-C₁₂)alkyl optionally interrupted by oxygen, and/or optionally substituted by aryl, cycloalkyl, heterocyclyl or one or more halogen (F, Cl, Br, I) atoms; further R₈ represents (C₃-C₆)cycloalkyl, hydroxy(C₁-C₁₂)alkyl, (C₁-C₁₂)alkoxy, (C₃-C₆)cycloalkoxy, aryl, heterocyclyl, (C₁-C₁₂)alkylsulfinyl, (C₁-C₁₂)alkylsulfonyl, (C₁-C₁₂)alkylthio, (C₃-C₆)cycloalkylthio, arylsulfinyl, arylsulfonyl, arylthio, aryl(C₁-C₁₂)alkylthio, aryl(C₁-C₁₂)alkylsulfonyl, arylC₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₂)alkylthio, (C₃-C₆)cycloalkyl(C₁-C₁₂)alkylsulfinyl or (C₃-C₆)cycloalkyl(C₁-C₁₂)alkylsulfonyl;

R₉ represents H or (C₁-C₁₂)alkyl;

R₁₀ represents H or (C₁-C₁₂)alkyl;

Q represents an unsubstituted or monosubstituted or polysubstituted (C₁-C₄)alkylene group, optionally interrupted by one or more groups/atoms selected among (C₃-C₇)cycloalkylene and a heteroatom being N, O and S, wherein any substituents each individually and independently are selected from (C₁-C₆)alkyl, (C₁-C₆)alkoxyl, oxy-(C₁-C₆)alkyl, (C₂-C₆)alkenyl, (C₂-C₆)alkynyl, (C₃-C₆)cycloalkyl, (C₃-C₆)cycloalkyl(C₁-C₄)alkylene, carboxyl, carboxy-(C₁-C₄)alkylene, aryl, aryl(C₁-C₄)alkylene, heterocyclyl, heterocyclyl(C₁-C₄)alkylene, nitro, cyano, halogeno (F, Cl, Br, I), hydroxyl, NR^{a(Q)}R^{b(Q)} in which R^{a(Q)} and R^{b(Q)} individually and independently from each other represents hydrogen, (C₁-C₄)alkyl or R^{a(Q)} and R^{b(Q)} together with the nitrogen atom represent piperidine, pyrrolidine, azetidine or aziridine, with the proviso that any substituents are connected to Q in such a way that no quarternary ammonium compounds are formed (by these connections); Further Q represents an unsubstituted or monosubstituted or polysubstituted (C₃-C₇)cycloalkylene wherein any substituents each individually and independently are selected from (C₁-C₆)alkyl, (C₁-C₆)alkoxyl, oxy-(C₁-C₆)alkyl, (C₂-C₆)alkenyl, (C₂-C₆)alkynyl, (C₃-C₆)cycloalkyl, (C₃-C₆)cycloalkyl(C₁-C₄)alkylene, carboxyl, carboxy-(C₁-C₄)alkylene, aryl, aryl(C₁-C₄)alkylene, heterocyclyl, heterocyclyl(C₁-C₄)alkylene, nitro, cyano, halogeno (F, Cl, Br, I), hydroxyl, NR^{a(Q)}R^{b(Q)} in which R^{a(Q)} and R^{b(Q)} individually and independently from each other represents hydrogen, (C₁-C₄)alkyl or R^{a}(Q) and R^{b(Q)} together with the nitrogen atom represent piperidine, pyrrolidine, azetidine or aziridine; Further Q represents aryl wherein any substituents each individually and independently are selected from (C₁-C₆)alkyl, (C₁-C₆)alkoxyl, oxy-(C₁-C₆)alkyl, (C₂-C₆)alkenyl, (C₂-C₆)alkenyl, (C₃-C₆)cycloalkyl, (C₃-C₆)cycloalkyl(C₁-C₄)alkylene, carboxyl, carboxy-(C₁-C₄)alkylene, aryl, aryl(C₁-C₄)alkylene, heterocyclyl, heterocyclyl(C₁-C₄)alkylene, nitro, cyano, halogeno (F, Cl, Br, I), hydroxyl, NR^{a(Q)}R^{b(Q)} in which R^{a(Q)} and R^{b(Q)} individually and independently from each other represents hydrogen, (C₁-C₄)alkyl or R^{a(Q)} and R^{b(Q)} together with the nitrogen atom represent piperidine, pyrrolidine, azetidine or aziridine;

R₁₆ represents (C₁-C₁₂)alkyl optionally interrupted by oxygen and/or optionally substituted by OH, aryl, cycloalkyl, heterocyclyl or one or more halogen (F, Cl, Br, I) atoms; further R₁₆ represents (C₃-C₆)cycloalkyl, hydroxy(C₂-C₁₂)alkyl, (C₁-C₁₂)alkoxy, (C₃-C₆)cycloalkoxy, aryl or heterocyclyl;

R₁₇ represents (C₁-C₂)alkyl optionally interrupted by oxygen and/or optionally substituted by OH, aryl, cycloalkyl, heterocyclyl or one or more halogen (F, Cl, Br, I) atoms; further R₇ represents (C₃-C₆)cycloalkyl, hydroxy(C₁-C₁₂)alkyl, (C₁-C₁₂)alkoxy, (C₃-C₆)cycloalkoxy, aryl or heterocyclyl;

R₁₈ represents (C₁-C₂)alkyl optionally interrupted by oxygen and/or optionally is substituted by OH, aryl, cycloalkyl, heterocyclyl or one or more halogen (F, Cl, Br, I) atoms; further R₁₈ represents (C₃-C₆)cycloalkyl, hydroxy(C₁-C₁₂)alkyl, (C₁-C₁₂)alkoxy, (C₃-C₆)cycloalkoxy, aryl or heterocyclyl;

R^{c} is absent or represents an unsubstituted or monosubstituted or polysubstituted (C₁-C₄)alkylene group, (C₁-C₄)oxoalkylene group, (C₁-C₄)alkyleneoxy or oxy-(C₁-C₄)alkylene group, wherein any substituents each individually and independently are selected from (C₁-C₄)alkyl, (C₁-C₄)alkoxyl, oxy-(C₁-C₄)alkyl, (C₂-C₄)alkenyl, (C₂-C₄)alkynyl, (C₃-C₆)cycloalkyl, carboxyl, carboxy-(C₁-C₄)alkyl, aryl, heterocyclyl, nitro, cyano, halogeno (F, Cl, Br, I), hydroxyl, NR^{a(Rc)}R^{b(Rc)} in which R^{a(Rc)} and R^{b(Rc)} individually and independently from each other represents hydrogen, (C₁-C₄)alkyl or R^{c(Rc)} and R^{b(Rc)} together with the nitrogen atom represent piperidine, pyrrolidine, azetidine or aziridine; Further R^{c} represents imino (—NH—), N-substituted imino (—NR₁₉—), (C₁-C₄)alkyleneimino or N-substituted (C₁-C₄)alkyleneimino (—N₁₉)—((C₁-C₄)alkylene) wherein the mentioned alkylene groups are unsubstituted or monosubstituted or polysubstituted with any substituents according to above; preferably R^{c} represents imino or (C₁-C₄)alkyleneimino or an unsubstituted or monosubstituted or polysubstituted (C₁-C₄)alkylene group or (C₁-C₄)oxoalkylene group with any substituents according to above;

R₁₉ represents H or (C₁-C₄)alkyl;

R^{d} represents (C₃-C₈)cycloalkyl, aryl or heterocyclyl, and anyone of these groups optionally substituted with one or more halogen (F, Cl, Br, I) atoms and/or one or more of the following groups, OH, CN, NO₂, (C₁-C₁₂)alkyl, (C₁-C₁₂)alkoxyC(O), (C₁-C₁₂)alkoxy, halogen substituted (C₁-C₁₂)alkyl, (C₃-C₆)cycloalkyl, aryl, heterocyclyl, (C₁-C₁₂)alkylsulfinyl, (C₁-C₁₂)alkylsulfonyl, (C₁-C₁₂)alkylthio, (C₃-C₆)cycloalkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₂)alkylsulfonyl, (C₃-C₆)cycloalkyl(C₁-C₁₂)alkylthio, (C₃-C₆)cycloalkyl(C₁-C₁₂)alkylsulfinyl, (C₃-C₆)cycloalkyl(C₁-C₁₂)alkylsulfonyl or a group of formula NR^{a(Rd)}R^{b(Rd)} in which R^{a(Rd)} and R^{b(Rd)} independently represent H, (C₁-C₁₂)alkyl, (C₁-C₁₂)alkylC(O) or R^{a(Rd)} and R^{b(Rd)} together with the nitrogen atom represent piperidine, pyrrolidine, azetidine or aziridine.

Preferred values as well as embodiments of each variable group or combinations thereof are as follows. Such values or embodiments may be used where appropriate with any of the values, definitions, claims, aspects or embodiments defined hereinbefore or hereinafter. In particular, each may be used as an individual limitation on the broadest definition as well as any other of the embodiments of formula (I).

For the avoidance of doubt it is to be understood that where in this specification a group is qualified by ‘hereinbefore defined’, ‘defined hereinbefore’ or ‘defined above’ the said group encompasses the first occurring and broadest definition as well as each and all of the particular definitions for that group.

It will be understood that when formula I compounds contain a chiral centre, the compounds of the invention may exist in, and be isolated in, optically active or racemic form. The invention includes any optically active or racemic form of a compound of formula I which act as P2Y₁₂ receptor antagonists. The synthesis of optically active forms may be carried out by standard techniques of organic chemistry well known in the art, for example by, resolution of a racemic mixture, by chiral chromatography, synthesis from optically active starting materials or by asymmetric synthesis.

It will also be understood that the compounds of the formula I may exhibit the phenomenon of tautomerism, the present invention includes any tautomeric form of a compound of formula I which is a P2Y₁₂ receptor antagonist.

It will also be understood that in so far as compounds of the present invention exist as solvates, and in particular hydrates, these are included as part of the present invention.

It is also to be understood that generic terms such as “alkyl” include both the straight chain and branched chain groups such as butyl and tert-butyl. However, when a specific term such as “butyl” is used, it is specific for the straight chain or “normal” butyl group, branched chain isomers such as “t-butyl” being referred to specifically when intended.

In one embodiment alkyl is unsubstituted or substituted by one or more halogen (F, Cl, Br, I) atoms and/or one or more of the following groups, OH, CN, NO₂, (C₁-C₁₂)alkyl, (C₁-C₁₂)alkoxyC(O), (C₁-C₁₂)alkoxy, halogen substituted (C₁-C₁₂)alkyl, (C₃-C₆)cycloalkyl, aryl, heterocyclyl, (C₁-C₁₂)alkylsulfinyl, (C₁-C₁₂)alkylsulfonyl, (C₁-C₁₂)alkylthio, (C₃-C₆)cycloalkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl, (C₃-C₆)cycloalkyl(C₁-C₁₂)alkylsulfonyl or a group of formula NR^{a}R^{b} in which R^{a} and R^{b} independently represent H, (C₁-C₁₂)alkyl, (C₁-C₁₂)alkylC(O) or R^{a} and R^{b} together with the nitrogen atom represent piperidine, pyrrolidine, azetidine or aziridine.

The term “alkyl” includes both linear or branched chain groups, optionally substituted by one or more halogens (F, Cl, Br, I) or mixed halogen atoms.

One embodiment of alkyl when substituted by one or more halogen atoms (F, Cl, Br, I) is, for example, alkyl substituted by one or more fluorine atoms. Another embodiment of halogen substituted alkyl includes perfluoroalkyl groups such as trifluoromethyl.

The term “cycloalkyl” generally denotes a substituted or unsubstituted (C₃-C₆), unless other chain length specified, cyclic hydrocarbon.

In one embodiment cycloalkyl is substituted by one or more halogen (F, Cl, Br, I) atoms and/or one or more of the following groups, OH, CN, NO₂, (C₁-C₁₂)alkyl, (C₁-C₁₂)alkoxyC(O), (C₁-C₁₂)alkoxy, halogen substituted (C₁-C₁₂)alkyl, (C₃-C₆)cycloalkyl, aryl, heterocyclyl, (C₁-C₁₂)alkylsulfinyl, (C₁-C₁₂)alkylsulfonyl, (C₁-C₁₂)alkylthio, (C₃-C₆)cycloalkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl, (C₃-C₆)cycloalkyl(C₁-C₁₂)alkylsulfonyl or a group of formula NR^{a}R^{b} in which R^{a} and R^{b} independently represent H, (C₁-C₁₂)alkyl, (C₁-C₁₂)alkylC(O) or R^{a} and R^{b} together with the nitrogen atom represent piperidine, pyrrolidine, azetidine or aziridine.

The term “alkoxy” includes both linear or branched chain groups, optionally substituted by one or more halogens (F, Cl, Br, I) or mixed halogen atoms.

The term aryl denotes a substituted or unsubstituted (C₆-C₁₄) aromatic hydrocarbon and includes, but is not limited to, phenyl, naphthyl, tetrahydronaphthyl, indenyl, indanyl, anthracenyl, fenantrenyl, and fluorenyl.

In one embodiment aryl is substituted by one or more halogen (F, Cl, Br, I) atoms and/or one or more of the following groups, OR CN, NO₂, (C₁-C₁₂)alkyl, (C₁-C₁₂)alkoxyC(O), (C₁-C₁₂)alkoxy, halogen substituted (C₁-C₁₂)alkyl, (C₃-C₆)cycloalkyl, aryl, heterocyclyl, (C₁-C₁₂)alkylsulfinyl, (C₁-C₁₂)alkylsulfonyl, (C₁-C₁₂)alkylthio, (C₃-C₆)cycloalkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl, (C₃-C₆)cycloalkyl(C₁-C₁₂)alkylsulfonyl or a group of formula NR^{a}R^{b} in which R^{a} and R^{b} independently represent H, (C₁-C₁₂)alkyl, (C₁-C₁₋₂)alkylC(O) or R^{a} and R^{b} together with the nitrogen atom represent piperidine, pyrrolidine, azetidine or aziridine.

The term “heterocyclyl” denotes a substituted or unsubstituted, 4- to 10-membered monocyclic or multicyclic ring system in which one or more of the atoms in the ring or rings is an element other than carbon, for example nitrogen, oxygen or sulfur, especially 4-, 5- or 6-membered aromatic or aliphatic heterocyclic groups, and includes, but is not limited to azetidine, furan, thiophene, pyrrole, pyrroline, pyrrolidine, dioxolane, oxathiolane, oxazolane, oxazole, thiazole, imidazole, imidazoline, imidazolidine, pyrazole, pyrazoline, pyrazolidine, isothiazole, oxadiazole, furazan, triazole, thiadiazole, pyran, pyridine as well as pyridine-N-oxide, piperidine, dioxane, morpholine, dithiane, oxathiane, thiomorpholine, pyridazine, pyrimidine, pyrazine, piperazine, triazine, thiadiazine, dithiazine, azaindole, azaindoline, indole, indoline, naphthyridine, benzoxadiazole, dihydrobenzodioxin, benzothiophene, benzothiadiazole, imidazothiazole, 2,3-dihydrobenzofuran, isoxazole, 3-benzisoxazole, 1,2-benzisoxazole, dihydropyrazole groups, and shall be understood to include all isomers of the above identified groups. For the above groups, e.g. azetidinyl, the term “azetidinyl” as well as “azetidinylene”, etc., shall be understood to include all possible regio isomers. It is further to be understood that the term heterocyclyl may be embodified by one selection among the given possible embodiments for a variable and embodified by another (or the same) selection for another variable, eg. R₄ when selected as heterocyclyl may be a furan, when R^{d} (also when selected as heterocyclyl) may be a pyrrole.

In one embodiment heterocyclyl is substituted by one or more halogen (F, Cl, Br, I) atoms and/or one or more of the following groups, OH, CN, NO₂, (C₁-C₁₂)alkyl, (C₁-C₁₂)alkoxyC(O), (C₁-C₁₂)alkoxy, halogen substituted (C₁-C₁₂)alkyl, (C₃-C₆)cycloalkyl, aryl, heterocyclyl, (C₁-C₁₂)alkylsulfinyl, (C₁-C₁₂)alkylsulfonyl, (C₁-C₁₂)alkylthio, (C₃-C₆)cycloalkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloakyl(C₁-C₁₂)alkylthio, (C₃-C₆)cycloalkyl(C₁-C₁₂)alkylsufinyl, (C₃-C₆)cycloalkyl(C₁-C₁₂)alkylsulfonyl or a group of formula NR^{a}R^{b} in which R^{a} and R^{b} independently represent H, (C₁-C₁₂)alkyl, (C₁-C₁₂)alkylC(O) or R^{a} and R^{b} together with the nitrogen atom represent piperidine, pyrrolidine, azetidine or aziridine.

In another embodiment of the invention the heterocyclyl group comprises an aromatic 5-membered or 6-membered heterocyclic ring containing one, two or three heteroatoms selected from nitrogen, oxygen and sulphur, and an aromatic 5-membered or 6-membered heterocyclic ring containing one, two or three heteroatoms selected from nitrogen, oxygen and sulphur which is fused to a benzene ring;

In an alternative embodiment of the invention the heterocyclyl group is a non-aromatic 5-membered or 6-membered heterocyclic ring containing one, two or three heteroatoms selected from nitrogen, oxygen and sulphur, fused to a benzene ring.

In a further embodiment of the invention the heterocyclyl group is a group chosen among furyl, pyrrolyl, thienyl, pyridyl, N-oxido-pyridyl, pyrazinyl, pyrimidinyl, pyridazinyl, imidazolyl, oxazolyl, isooxazolyl, thiazolyl, isothiazolyl, oxadiazolyl, 1,2,3-triazolyl, 1,2,4-triazolyl, benzofuranyl, quinolyl, isoquinolyl, benzimidazolyl, indolyl, benzodihydrofuranyl, benzodioxolyl (such as 1,3-benzodioxolyl), benzoxadiazole, dihydrobenzodioxin, benzothiophene, benzothiadiazole, imidazothiazole, 2,3-dihydrobenzofuran, isoxazole, dihydropyrazole and benzodioxanyl (such as 1,4-benzodioxanyl). More particular values include, for example, furyl, pyrrolyl, thienyl, pyridyl, pyrazinyl, pyrimidinyl, pyridazinyl, benzoxadiazole, dihydrobenzodioxin, benzothiophene, benzothiadiazole, imidazothiazole, 2,3-dihydrobenzofuran, isoxazole, 1,2-benzisoxazole, dihydropyrazole and benzdioxanyl (such as 1,4-benzdioxanyl).

In an even further embodiment of the invention the heterocyclyl group is a group chosen among furyl, pyrrolyl, thienyl, pyridyl, N-oxido-pyridyl, pyrazinyl, pyrimidinyl, pyridazinyl, benzoxadiazole, dihydrobenzodioxin, benzothiophene, benzothiadiazole, imidazothiazole, 2,3-dihydrobenzofuran, isoxazole, 1,2-benzisoxazole or dihydropyrazole.

In one embodiment of the invention R₁ represents R₆OC(O).

In another embodiment of the invention R₁ represents R₁₆SC(O).

In yet another embodiment R₁ represents a group (gII),

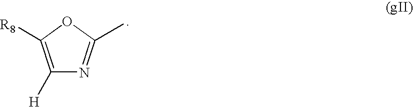

In a further embodiment of the invention R₁ is selected among R₆OC(O) and R₁₆SC(O) wherein R₆ can be methyl, ethyl, 2-hydroxyethyl, 2,2,2-trifluoroethyl, isopropyl, cyclo-propyl, iso-butyl, n-butyl, cyclo-butyl, n-propyl, tertbutyl, cyclo-pentyl, 2,2-dimethylpropyl, benzyl and 4-fluorobenzyl and wherein R₁₆ is ethyl.

R₁ may also be embodified by the group gII,

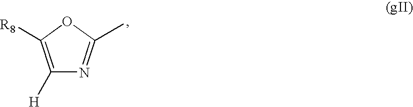

in which R₈ is selected from H, (C₁-C₆)alkyl, such as methyl or ethyl.

In another embodiment for the group R₈ this group can be chosen among hydrogen, methyl, ethyl, n-propyl and m-butyl.

Embodiments for R₂ include, for example, H and(C₁-C₄)alkyl. Other embodiments for R₂ are methyl, ethyl, iso-propyl, phenyl, methoxy, or amino unsubstituted or optionally substituted with methyl.

A special embodiment for R₂ is (C₁-C₄)alkyl.

In another embodiment R₂ is represented by phenyl, methoxy or amino unsubstituted or optionally substituted with methyl.

In an alternative embodiment R₂ is represented by (C₁-C₄)alkyl, phenyl, methoxy or amino unsubstituted or optionally substituted with methyl.

In an even further alternative embodiment R₂ is represented by (C₁-C₄)alkyl, phenyl or methoxy.

Embodiments for R₃ include, for example, H, methyl, methylsulfinyl, hydroxymethyl, methoxy or amino unsubstituted or optionally substituted with one or two methyl groups.

Other embodiments for R₃ include H or amino unsubstituted or optionally substituted with one or two methyl groups.

Embodiments for R₄ include H, halogen such as chloro, methyl, cyano, nitro, amino unsubstituted or optionally substituted with one or two methyl groups and further includes 4-methoxy-4-oxobutoxy, 3-carboxy-propoxy and methylcarbonyl.

Further embodiments for R₈ include, hydrogen, methyl and ethyl.

In one preferred embodiment Q represents an unsubstituted or monosubstituted or polysubstituted (C₁-C₄)alkylene group, optionally interrupted by one or more groups/atoms selected among (C₃-C₇)cycloalkylene and a heteroatom being N, O and S, wherein any substituents each individually and independently are selected from (C₁-C₆)alkyl, (C₁-C₆)alkoxyl, oxy-(C₁-C₆)alkyl, (C₂-C₆)alkenyl, (C₂-C₆)alkynyl, (C₃-C₆)cycloalkyl, (C₃-C₆)cycloalkyl(C₁-C₄)alkylene, carboxyl, carboxy-(C₁-C₄)alkylene, aryl, aryl(C₁-C₄)alkylene, heterocyclyl, heterocyclyl(C₁-C₄)alkylene, nitro, cyano, halogeno (F, Cl, Br, I), hydroxyl, NR^{a(Q)}R^{b(Q)} in which R^{a(Q)} and R^{b(Q)} individually and independently from each other represents hydrogen, (C₁-C₄)alkyl or R^{a(Q)} and R^{b(Q)} together with the nitrogen atom represent piperidine, pyrrolidine, azetidine or aziridine, with the proviso that any substituents are connected to Q in such a way that no quarternary anumonium compounds are formed (by these connections); Further in the same embodiment Q represents an unsubstituted or monosubstituted or polysubstituted (C₃-C₇)cycloalkylene wherein any substituents each individually and independently are selected from (C₁-C₆)alkyl, (C₁-C₆)alkoxyl, oxy-(C₁-C₆)alkyl, (C₂-C₆)alkenyl, (C₂-C₆)alkynyl, (C₃-C₆)cycloalkyl, (C₃-C₆)cycloalkyl(C₁-C₄)alkylene, carboxyl, carboxy-(C₁-C₄)alkylene, aryl, aryl(C₁-C₄)alkylene, heterocyclyl, heterocyclyl(C₁-C₄)alkylene, nitro, cyano, halogeno (F, Cl, Br, I), hydroxyl, NR^{a(Q)}R^{b(Q)} in which R^{a(Q)} and R^{b(Q)} individually and independently from each other represents hydrogen, (C₁-C₄)alkyl or R^{a(Q)} and R^{b(Q)} together with the nitrogen atom represent piperidine, pyrrolidine, azetidine or aziridine;

Further embodiments for R^{d} includes aryl or heterocyclyl, more particularly, aryl or aromatic heterocyclyl.

Another embodiment for R^{d} include, aryl such as phenyl and aromatic heterocyclyl such as thienyl.

Other embodiments of R^{d} include phenyl which optionally may be substituted.

In a special embodiment R^{d} represents aryl, heterocyclyl or (C₃-C₆)cycloalkyl, and anyone of these groups are optionally substituted with one or more halogen (F, Cl, Br, I) atoms or mixed halogen atoms, and/or one or more of the following groups, OH, CN, NO₂, (C₁-C₁₂)alkyl, (C₁-C₂)alkoxyC(O), (C₁-C₁₂)alkoxy, halogen substituted (C₁-C₁₂)alkyl, (C₃-C₆)cycloalkyl, aryl, heterocyclyl, (C₁-C₁₂)alkylsulfinyl, (C₁-C₁₂)alkylsulfonyl, (C₁-C₁₂)alkylthio, (C₃-C₆)cycloalkylthio, arylsulfinyl, arylsulfonyl, arylthio, aryl(C₁-C₁₂)alkylthio, aryl(C₁-C₁₂)alkylsulfonyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)Cycloalkyl(C₁-C₁₂)alkylthio, (C₃-C₆)cycloalkyl(C₁-C₁₂)alkylsulfinyl, (C₃-C₆)cycloalkyl(C₁-C₁₂)alkylsulfonyl or a group of formula NR^{b(Rd)}R^{b(Rd)} in which R^{a(Rd)} and R^{b(Rd)} independently represent H, (C₁-C₁₂)alkyl, (C₁-C₁₂)alkylC(O) or R^{a(Rd)} and R^{b(Rd)} together with the nitrogen atom represent piperidine, pyrrolidine, azetidine or aziridine;

Even further embodiments for R^{d} include phenyl optionally substituted at the 2,3,4 or 5-positions as well as any combination thereof. Example of substituents are cyano, tetrazol-5-yl, methoxy, trifluoromethoxy, methyl, trifluoromethyl, fluoro, chloro, bromo, methylsulfonyl, nitro, 3-methyl-5-oxo-4,5-dihydro-1H-pyrazol-1-yl. Two adjacent positions (e.g. 2,3) may also be connected to form a ring. Example of such a substituent is 2-naphtyl. Further more specific values for heteroaryls are 2-chloro-5-thienyl, 3-bromo-5-chloro-2-thienyl, 2,1,3-benzoxadiazol-4-yl, 2,4-dimethyl-1,3-thiazol-5-yl, 2,3-dihydro-1,4-benzodioxin-6-yl, 5-chloro-3-methyl-1-benzothien-2-yl, 2,1,3-benzothiadiazol-4-yl, 2,5-dimethyl-3-furyl, 6-chloroimidazo[2,1-b][1,3]thiazol-5-yl, 2,3-dihydro-1-benzofuran-5-yl, 5-chloro-3-thienyl, 5-isoxazol-5-yl-2-thienyl, 5-isoxazol-3-yl-2-thienyl, 4-bromo-5-chloro-2-thienyl, 5-bromo-6-chloropyridin-3-yl, S-bromo-2-thienyl, 5-pyridin-2-yl-2-thienyl, 2,5-dichloro-3-thienyl, 4,5-dichloro-2-thienyl, benzothien-3-yl, 2,5-dimethyl-3-thienyl, 3-thienyl, 2-thienyl, 5-methylisoxazol-4-yl, pyridin-3-yl, [1-methyl-5-(trifluoromethyl)-1H-pyrazol-3-yl]-2-thienyl, 5-chloro-1,3-dimethyl-1H-pyrazol-4-yl, 4-[(4-chlorophenyl)sulfonyl]-3-methyl-2-thienyl, 5-(methoxycarbonyl)-2-furyl and 4-(methoxycarbonyl)-5-methyl-2-furyl.

In one embodiment of the invention R^{c} is absent or represents an unsubstituted or monosubstituted or disubstituted (C₁-C₄)alkylene group wherein any substituents each individually and independently are selected from (C₁-C₄)alkyl, (C₁-C₄)alkoxyl, oxy-(C₁-C₄)alkyl, (C₂-C₄)alkenyl, (C₂-C₄)alkynyl, (C₃-C₆)cycloalkyl, carboxyl, carboxy-(C₁-C₄)alkyl, aryl, heterocyclyl, nitro, cyano, halogeno (F, Cl, Br, I), hydroxyl, NR^{a(Rc)}R^{b(Rc)} in which R^{a(Rc)} and R^{b(Rc)} individually and independently from each other represents hydrogen, (C₁-C₄)alkyl or R^{a(Rc)} and R^{b(Rc)} together with the nitrogen atom represent piperidine, pyrrolidine, azetidine or aziridine, and R^{d} represents aryl.

In a preferred embodiment of the invention R^{c} is absent or represents an unsubstituted or monosubstituted or disubstituted (C₁-C₃)alkylene group wherein any substituents each individually and independently are selected from (C₁-C₄)alkyl, (C₁-C₄)alkoxyl, oxy-(C₁-C₄)alkyl, (C₂-C₄)alkenyl, (C₂-C₄)alkyl, (C₃-C₆)cycloalkyl, carboxyl, carboxy-(C₁-C₄)alkyl, aryl, heterocyclyl, nitro, cyano, halogeno (F, Cl, Br, I), hydroxyl, NR^{a(Rc)}R^{b(Rc)} in which R^{a(Rc)} and R^{b(Rc)} individually and independently from each other represents hydrogen, (C₁-C₄)alkyl or R^{a(Rc)} and R^{b(Rc)} together with the nitrogen atom represent piperidine, pyrrolidine, azetidine or aziridine, and R^{d} represents aryl.

In a further embodiment of the invention R^{c} is absent or represents an unsubstituted or monosubstituted or disubstituted (C₁-C₄)alkylene group wherein any substituents each individually and independently are selected from (C₁-C₄)alkyl, (C₁-C₄)alkoxyl, oxy-(C₁-C₄)alkyl, (C₂-C₄)alkenyl, (C₂-C₄)alkynyl, (C₃-C₆)cycloalkyl, carboxyl, carboxy-(C₁-C₄)alkyl, aryl, heterocyclyl, nitro, cyano, halogeno (F, Cl, Br, I), hydroxyl, NR^{a(Rc)}R^{b(Rc)} in which R^{a(Rc)} and R^{b(Rc)} individually and independently from each other represents hydrogen, (C₁-C₄)alkyl or R^{a(Rc)} and R^{b(Rc)} together with the nitrogen atom represent piperidine, pyrrolidine, azetidine or aziridine, and R^{d} represents heterocyclyl.

In a further preferred embodiment of the invention R^{c} is absent or represents an unsubstituted or monosubstituted or disubstituted (C₁-C₃)alkylene group wherein any substituents each individually and independently are selected from (C₁-C₄)alkyl, (C₁-C₄)alkoxy, oxy-(C₁-C₄)alkyl, (C₂-C₄)alkenyl, (C₂-C₄)alkynyl, (C₃-C₆)cycloalkyl, carboxyl, carboxy-(C₁-C₄)alkyl, aryl, heterocyclyl, nitro, cyano, halogeno (F, Cl, Br, I), hydroxyl, NR^{a(Rc)}R^{b(Rc)} in which R^{a(Rc)} and R^{b(Rc)} individually and independently from each other represents hydrogen, (C₁-C₄)alkyl or R^{a(Rc)} and R^{b(Rc)} together with the nitrogen atom represent piperidine, pyrrolidine, azetidine or aziridine, and R^{d} represents heterocyclyl.

In a particular embodiment of the invention R^{c} is absent or represents a C₁-alkylene group wherein any substituents each individually and independently are selected from (C₁-C₄)alkyl, (C₁-C₄)alkoxy, oxy-(C₁-C₄)alkyl, (C₂-C₄)alkenyl, (C₂-C₄)alkynyl, (C₃-C₆)cycloalkyl, carboxyl, carboxy-(C₁-C₄)alkyl, aryl, heterocyclyl, nitro, cyano, halogeno (F, Cl, Br, I), hydroxyl, NR^{a(Rc)}R^{b(Rc)} in which R^{a(Rc)} and R^{b(Rc)} individually and independently from each other represents hydrogen, (C₁-C₄)alkyl or R^{a(Rc)} and R^{b(Rc)} together with the nitrogen atom represent piperidine, pyrrolidine, azetidine or aziridine, and R^{d} represents aryl.

In one embodiment of the invention R₁₉ represents hydrogen.

In another embodiment of the invention R₁₉ represents methyl.

In a most particular embodiment of the invention R^{c}R^{d} represents a benzyl group, or a benzyl group which is substituted according to what is described in connection to substitution of the aryl group.

A 2nd embodiment of formula I is defined by;

R₁ represents R₆OC(O), R₇C(O), R₁₆SC(O), R₁₇S, R₁₈C(S) or a group gII,

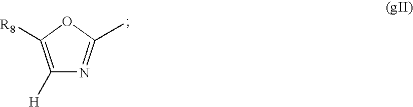

R₂ represents H, CN, NO₂, (C₁-C₆)alkyl optionally interrupted by oxygen and/or optionally substituted by OH, aryl, cycloalkyl, heterocyclyl or one or more halogen (F, Cl, Br, I) atoms; further R₂ represents (C₁-C₆)alkoxy optionally substituted by one or more halogen (F, Cl, Br, I) atoms; further R₂ represents (C₃-C₆)cycloalkyl, hydroxy(C₁-C₆)alkyl, is (C₁-C₆)alkylC(O), (C₁-C₆)alkylthioC(O), (C₁-C₆)alkylC(S), (C₁-C₆)alkoxyC(O), (C₃-C₆)cycloalkoxy, aryl, arylC(O), aryl(C₁-C₆)alkylC(O), heterocyclyl, heterocyclylC(O), heterocyclyl(C₁-C₆)alkylC(O), (C₁-C₆)alkylsulfinyl, (C₁-C₆)alkylsulfonyl, (C₁-C₆)alkylthio, (C₃-C₆)cycloalkylthio, arylsulfinyl, arylsulfonyl, arylthio, aryl(C₁-C₆)alkylthio, aryl(C₁-C₆)alkylsulfinyl, aryl(C₁-C₆)alkylsulfonyl, heterocyclyl(C₁-C₆)alkylthio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkylsulfinyl, (C₃-C₆)cycloalkyl(C₁-C₆)alkylsulfonyl or a group of formula NR^{a(2)}R^{b(2)} in which R^{a(2)} and R^{b(2)} independently represent H, (C₁-C₆)alkyl, (C₁-C₆)alkylC(O) or R^{a(2)} and R^{b(2)} together with the nitrogen atom represent piperidine, pyrrolidine, azetidine or aziridine;

R₃ represents H, CN, NO₂, halogen (F, Cl, Br, I), (C₁-C₆)alkyl optionally interrupted by oxygen and/or optionally substituted by OH, aryl, cycloalkyl, heterocyclyl or one or more halogen atoms; further R₃ represents (C₁-C₆)alkoxy optionally substituted by one or more halogen (F, Cl, Br, I) atoms; further R₃ represents (C₃-C₆)cycloalkyl, hydroxy(C₁-C₆)alkyl, (C₁-C₆)alkylC(O), (C₁-C₆)alkylthioC(O), (C₁-C₆)alkylC(S), (C₁-C₆)alkoxyC(O), (C₃-C₆)cycloalkoxy, aryl, arylC(O), aryl(C₁-C₆)alkylC(O), heterocyclyl, heterocyclylC(O), heterocyclyl(C₁-C₆)alkylC(O), (C₁-C₆)alkylsulfinyl, (C₁-C₆)alkylsulfonyl, (C₁-C₆)alkylthio, (C₃-C₆)cycloalkylthio, arylsulfinyl, arylsulfonyl, arylthio, aryl(C₁-C₆)alkylthio, aryl(C₁-C₆)alkylsulfinyl, aryl(C₁-C₆)alkylsulfonyl, heterocyclyl(C₁-C₆)alkylthio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkylsulfanyl, (C₃-C₆)cycloalkyl(C₁-C₆)alkylsulfonyl or a group of formula NR^{a(3)}R^{b(3)} in which R^{a(3)} and R^{b(3)} independently represent H, (C₁-C₆)alkyl, (C₁-C₆)alkylC(O) or R^{a(3)} and R^{b(3)} together with the nitrogen atom represent piperidine, pyrrolidine, azetidine or aziridine;
R₄ represents H, CN, NO₂, halogen (F, Cl, Br, I), (C₁-C₆)alkyl optionally interrupted by oxygen and/or optionally substituted by OH, COOH, (C₁-C₆)alkoxycarbonyl, aryl, cycloalkyl, heterocyclyl or one or more halogen atoms; further R₄ represents (C₃-C₆)cycloalkyl, hydroxy(C₁-C₆)alkyl, (C₁-C₆)alkylC(O), (C₁-C₆)alkoxy wherein the alkoxy group may optionally be substituted by one or more halogen (F, Cl, Br, I) atoms, OH and/or COOH and/or (C₁-C₃)alkoxycarbonyl; further R₄ represents (C₁-C₆)alkylthioC(O), (C₁-C₆)alkylC(S), (C₁-C₆)alkoxyC(O), (C₃-C₆)cycloalkoxy, aryl, arylC(O), aryl(C₁-C₆)alkylC(O), heterocyclyl, heterocyclylC(O), heterocyclyl(C₁-C₆)alkylC(O), (C₁-C₆)alkylsulfinyl, (C₁-C₆)alkylsulfonyl, (C₁-C₆)alkylthio, (C₃-C₆)cycloalkylthio, arylsulfinyl, arylsulfonyl, arylthio, aryl(C₁-C₆)alkylthio, aryl(C₁-C₆)alkylsulfinyl, aryl(C₁-C₆)alkylsulfonyl, heterocyclyl(C₁-C₆)alkylthio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkylsulfinyl, (C₃-C₆)cycloalkyl(C₁-C₆)alkylsulfonyl or a group of formula NR^{a(4)}R^{b(4)} in which R^{a(4)} and R^{b(4)} independently represent H, (C₁-C₆)alkyl, (C₁-C₆)alkylC(O) or R^{a(4)} and R^{b(4)} together with the nitrogen atom represent piperidine, pyrrolidine, azetidine or aziridine;

R₆ represents (C₁-C₆)alkyl optionally interrupted by oxygen, (with the proviso that any such oxygen must be at least 1 carbon atom away from the ester-oxygen connecting the R₆ group) and/or optionally substituted by OH, aryl, cycloalkyl, heterocyclyl or one or more halogen (F, Cl, Br, I) atoms; further R₆ represents (C₃-C₆)cycloalkyl, hydroxy(C₂-C₆)alkyl, aryl or heterocyclyl;

R₇ represents (C₁-C₆)alkyl optionally interrupted by oxygen, and/or optionally substituted by OH, aryl, cycloalkyl, heterocyclyl or one or more halogen (F, Cl, Br, I) atoms; further R₇ represents (C₃-C₆)cycloalkyl, hydroxy(C₁-C₆)alkyl, aryl or heterocyclyl;

R₈ represents H, (C₁-C₆)alkyl optionally interrupted by oxygen, and/or optionally substituted by aryl, cycloalkyl, heterocyclyl or one or more halogen (F, Cl, Br, I) atoms; further R₃ represents (C₃-C₆)cycloalkyl, hydroxy(C₁-C₆)alkyl, (C₁-C₆)alkoxy, (C₃-C₆)cycloalkoxy, aryl, heterocyclyl, (C₁-C₆)alkylsulfinyl, (C₁-C₆)alkylsulfonyl, (C₁-C₆)alkylthio, (C₃-C₆)cycloalkylthio, arylsulfinyl, arylsulfonyl, arylthio, aryl(C₁-C₆)alkylthio, aryl(C₁-C₆)alkylsulfinyl, aryl(C₁-C₆)alkylsulfonyl, heterocyclyl(C₁-C₆)alkylthio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkylsulfinyl or (C₃-C₆)cycloalkyl(C₁-C₆)alkylsulfonyl;

R₉ represents H or (C₁-C₆)alkyl;

R₁₀ represents H or (C₁-C₆)alkyl;

Q represents an unsubstituted or monosubstituted or polysubstituted (C₁-C₄)alkylene group, optionally interrupted by one or more groups/atoms selected among (C₃-C₇)cycloalkylene and a heteroatom being N, O and S, wherein any substituents each individually and independently are selected from (C₁-C₆)alkyl, (C₁-C₆)alkoxyl, oxy-(C₁-C₆)alkyl, (C₂-C₆)alkenyl, (C₂-C₆)alkynyl, (C₃-C₆)cycloalkyl, (C₃-C₆)cycloalkyl(C₁-C₄)alkylene, carboxyl, carboxy-(C₁-C₄)alkylene, aryl, aryl(C₁-C₄)alkylene, heterocyclyl, heterocyclyl(C₁-C₄)alkylene, nitro, cyano, halogeno (F, Cl, Br, I), hydroxyl, NR^{a(Q)}R^{b(Q)} in which R^{a(Q)} and R^{b(Q)} individually and independently from each other represents hydrogen, (C₁-C₄)alkyl or R^{a(Q)} and R^{b(Q)} together with the nitrogen atom represent piperidine, pyrrolidine, azetidine or aziridine, with the proviso that any substituents are connected to Q in such a way that no quarternary ammonium compounds are formed (by these connections); Further Q represents an unsubstituted or mono substituted or polysubstituted (C₃-C₇)cycloalkylene wherein any substituents each individually and independently are selected from (C₁-C₆)alkyl, (C₁-C₆)alkoxyl, oxy-(C₁-C₆)alkyl, (C₂-C₆)alkenyl, (C₂-C₆)alkynyl, (C₃-C₆)cycloalkyl, (C₃-C₆)cycloakyl(C₁-C₄)alkylene, carboxyl, carboxy-(C₁-C₄)alkylene, aryl, aryl(C₁-C₄)alkylene, heterocyclyl, heterocyclyl(C₁-C₄)alkylene, nitro, cyano, halogeno (F, Cl, Br, I), hydroxyl, NR^{a(Q)}R^{b(Q)} in which R^{a(Q)} and R^{b(Q)} individually and independently from each other represents hydrogen, (C₁-C₄)alkyl or R^{a(Q)} and R^{b(Q)} together with the nitrogen atom represent piperidine, pyrrolidine, azetidine or aziridine; Further Q represents aryl wherein any substituents each individually and independently are selected from (C₁-C₆)alkyl, (C₁-C₆)alkoxyl, oxy-(C₁-C₆)alkyl, (C₂-C₆)alkenyl, (C₂-C₆)alkyl, (C₃-C₆)cycloalkyl, (C₃-C₆)cycloalkyl(C₁-C₄)alkylene, carboxyl, carboxy-(C₁-C₄)alkylene, aryl, aryl(C₁-C₄)alkylene, heterocyclyl, heterocyclyl(C₁-C₄)alkylene, nitro, cyano, halogeno (F, Cl, Br, I), hydroxyl, NR^{a(Q)}R^{b(Q)} in which R^{a(Q)} and R^{b(Q)} individually and independently from each other represents hydrogen, (C₁-C₄)alkyl or R^{a(Q)} and R^{b(Q)} together with the nitrogen atom represent piperidine, pyrrolidine, azetidine or aziridine;

R₁₆ represents (C₁-C₆)alkyl optionally interrupted by oxygen and/or optionally substituted by OH, aryl, cycloalkyl, heterocyclyl or one or more halogen (F, Cl, Br, I) atoms; further R₁₄ represents (C₃-C₆)cycloalkyl, hydroxy(C₂-C₆)alkyl, (C₁-C₆)alkoxy, (C₃-C₆)cycloalkoxy, aryl, or heterocyclyl;

R₁₇ represents (C₁-C₆)alkyl optionally interrupted by oxygen and/or optionally substituted by OH, aryl, cycloalkyl, heterocyclyl or one or more halogen (F, Cl, Br, I) atoms; further R₁₇ represents (C₃-C₆)cycloalkyl, hydroxy(C₁-C₆)alkyl, (C₁-C₆)alkoxy, (C₃-C₆)cycloalkoxy, aryl or heterocyclyl;

R₁₈ represents (C₁-C₆)alkyl optionally interrupted by oxygen and/or optionally substituted by OH, aryl, cycloalkyl, heterocyclyl or one or more halogen (F, Cl, Br, I) atoms; further R¹⁸ represents (C₃-C₆)cycloalkyl, hydroxy(C₁-C₆)alkyl, (C₁-C₆)alkoxy, (C₃-C₆)cycloalkoxy, aryl or heterocyclyl;

R^{c} is absent or represents an unsubstituted or monosubstituted or polysubstituted (C₁-C₄)alkylene group, (C₁-C₄)oxyalkylene group, (C₁-C₄)alkyleneoxy or oxy-(C₁-C₄)alkylene group, wherein any substituents each individually and independently are selected from (C₁-C₄)alkyl, (C₁-C₄)alkoxyl, oxy-(C₁-C₄)alkyl, (C₂-C₄)alkenyl, (C₂-C₄)alkaryl, (C₃-C₆)cycloalkyl, carboxyl, carboxy-(C₁-C₄)alkyl, aryl, heterocyclyl, nitro, cyano, halogeno (F, Cl, Br, I), hydroxyl, NR^{a(Rc)}R^{b(Rc)} in which R^{a(Rc)} and R^{b(Rc)} individually and independently from each other represents hydrogen, (C₁-C₄)alkyl or R^{a(Rc)} and R^{b(Rc)} together with the nitrogen atom represent piperidine, pyrrolidine, azetidine or aziridine; Further R^{c} represents imino (—NH—), N-substituted imino (—NR₁₉—), (C₁-C₄)alkyleneimino or N-substituted (C₁-C₄)alkyleneimino (—N(R¹⁹)—((C₁-C₄)alkylene) wherein the mentioned alkylene groups are unsubstituted or monosubstituted or polysubstituted with any substituents according to above; preferably R^{c} represents imino or (C₁-C₄)alkyleneimino or an unsubstituted or monosubstituted or polysubstituted (C₁-C₄)alkylene group or (C₁-C₄)oxoalkylene group with any substituents according to above;

R₁₉ represents H or (C₁-C₄)alkyl;

R^{d} represents (C₃-C₈)cycloalkyl, aryl or heterocyclyl, and anyone of these groups optionally substituted with one or more halogen (F, Cl, Br, I) atoms and/or one or more of the following groups, OH, CN, NO₂, (C₁-C₆)alkyl, (C₁-C₆)alkoxyC(O), (C₁-C₆)alkoxy, halogen substituted (C₁-C₆)alkyl, (C₃-C₆)cycloalkyl, aryl, heterocyclyl, (C₁-C₆)alkylsulfinyl, (C₁-C₆)alkylsulfonyl, (C₁-C₆)alkylthio, (C₃-C₆)cycloalkylthio, arylsulfinyl, arylsulfonyl, arylthio, aryl(C₁-C₆)alkylthio, aryl(C₁-C₆)alkylsulfinyl, aryl(C₁-C₆)alkylsulfonyl, heterocyclyl(C₁-C₆)alkylthio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkylsulfinyl, (C₃-C₆)cycloalkyl(C₁-C₆)alkylsulfonyl or a group of formula NR^{a(Rd)}R^{b(Rd)} in which R^{a(Rd)} and R^{b(Rd)} independently represent H, (C₁-C₆)alkyl, (C₁-C₆)alkylC(O) or R^{a(Rd)} and R^{b(Rd)} together with the nitrogen atom represent piperidine, pyrrolidine, azetidine or aziridine.

A 3rd embodiment of formula I is defined by;

R₁ represents R₆OC(O), R₁₆SC(O), or a group gII,

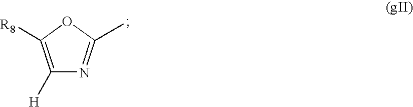

R₂ represents H, CN, NO₂, (C₁-C₆)alkyl optionally interrupted by oxygen and/or optionally substituted by OH, aryl, cycloalkyl, heterocyclyl or one or more halogen (F, Cl, Br, I) atoms; further R₂ represents (C₁-C₆)alkoxy optionally substituted by one or more halogen (F, Cl, Br, I) atoms; further R₂ represents (C₃-C₆)cycloalkyl, hydroxy(C₁-C₆)alkyl, (C₁-C₆)alkylC(O), (C₁-C₆)alkylthioC(O), (C₁-C₆)alkylC(S), (C₁-C₆)alkoxyC(O), (C₃-C₆)cycloalkoxy, aryl, arylC(O), aryl(C₁-C₆)alkylC(O), heterocyclyl, heterocyclylC(O), heterocyclyl(C₁-C₆)alkylC(O) or a group of formula NR^{a(2)}R^{b(2)} in which R^{a(2)} and R^{b(2)} independently represent H, (C₁-C₆)alkyl, (C₁-C₆)alkylC(O) or R⁽²⁾ and R^{b(2)} together with the nitrogen atom represent piperidine, pyrrolidine, azetidine or aziridine;

R₃ represents H, CN, NO₂, halogen (F, Cl, Br, I), (C₁-C₆)alkyl optionally interrupted by oxygen and/or optionally substituted by OH, aryl, cycloalkyl, heterocyclyl or one or more halogen atoms; further R₃ represents (C₁-C₆)alkoxy optionally substituted by one or more halogen (F, Cl, Br, I) atoms; further R₃ represents (C₃-C₆)cycloalkyl, hydroxy(C₁-C₆)alkyl, (C₁-C₆)alkylC(O), (C₁-C₆)alkylthioC(O), (C₁-C₆)alkylC(S), (C₁-C₆)alkoxyC(O), (C₃-C₆)cycloalkoxy, aryl, arylC(O), aryl(C₁-C₆)alkylC(O), heterocyclyl, heterocyclylC(O), heterocyclyl(C₁-C₆)alkylC(O), (C₁-C₆)alkylsulfinyl, or a group of formula NR^{a(3)}R^{b(3)} in which R^{a(3)} and R^{b(3)} independently represent H, (C₁-C₆)alkyl, (C₁-C₆)alkylC(O) or R^{a(3)} and R^{b(3)} together with the nitrogen atom represent piperidine, pyrrolidine, azetidine or aziridine;

R₄ represents H, CN, NO₂, halogen (F, Cl, Br, I), (C₁-C₆)alkyl optionally interrupted by oxygen and/or optionally substituted by OH, COOH, aryl, cycloalkyl, heterocyclyl or one or more halogen atoms; further R₄ represents (C₃-C₆)cycloalkyl, hydroxy(C₁-C₆)alkyl, (C₁-C₆)alkylC(O), (C₁-C₆)alkoxy wherein the alkoxy group may optionally be substituted by one or more halogen (F, Cl, Br, I) atoms, OH and/or COOH and/or methoxycarbonyl; further R₄ represents (C₁-C₆)alkylthioC(O), (C₁-C₆)alkylC(S), (C₁-C₆)alkoxyC(O), (C₃-C₆)cycloalkoxy, aryl, arylC(O), aryl(C₁-C₆)alkylC(O), heterocyclyl, heterocyclylC(O), heterocyclyl(C₁-C₆)alkylC(O) or a group of formula NR^{a(4)}R^{b(4)} in which R^{a(4)} and R^{b(4)} independently represent H, (C₁-C₆)alkyl, (C₁-C₆)alkylC(O) or R^{a(4)} and R^{b(4)} together with the nitrogen atom represent piperidine, pyrrolidine, azetidine or aziridine;

R₆ represents (C₁-C₆)alkyl optionally interrupted by oxygen, (with the proviso that any such oxygen must be at least 1 carbon atom away from the ester-oxygen connecting the R₆ group) and/or optionally substituted by OH, aryl, cycloalkyl, heterocyclyl or one or more halogen (F, Cl, Br, I) atoms; further R₆ represents (C₃-C₆)cycloalkyl, hydroxy(C₂-C₆)alkyl, aryl or heterocyclyl;

R₈ represents H, (C₁-C₆)alkyl optionally interrupted by oxygen, and/or optionally substituted by aryl, cycloalkyl, heterocyclyl or one or more halogen (F, Cl, Br, I) atoms; further R₈ represents (C₃-C₆)cycloalkyl, hydroxy(C₁-C₆)alkyl, (C₁-C₆)alkoxy, (C₃-C₆)cycloalkoxy, aryl or heterocyclyl;

R₉ represents H or (C₁-C₆)alkyl;

R₁₀ represents H or (C₁-C₆)alkyl;

Q represents an unsubstituted or monosubstituted or polysubstituted (C₁-C₄)alkylene group, optionally interrupted by one or more groups/atoms selected among (C₃-C₇)cycloalkylene and a heteroatom being N, O and S, wherein any substituents each individually and independently are selected from (C₁-C₆)alkyl, (C₁-C₆)alkoxyl, oxy-(C₁-C₆)alkyl, (C₂-C₆)alkenyl, (C₂-C₆)alkynyl, (C₃-C₆)cycloalkyl, (C₃-C₆)cycloalkyl(C₁-C₄)alkylene, carboxyl, carboxy-(C₁-C₄)alkylene, aryl, aryl(C₁-C₄)alkylene, heterocyclyl, heterocyclyl(C₁-C₄)alkylene, nitro, cyano, halogeno (F, Cl, Br, I), hydroxyl, NR^{a(Q)}R^{b(Q)} in which R^{a(Q)} and R^{b(Q)} individually and independently from each other represents hydrogen, (C₁-C₄)alkyl or R^{a(Q)} and R^{b(Q)} together with the nitrogen atom represent piperidine, pyrrolidine, azetidine or aziridine, with the proviso that any substituents are connected to Q in such a way that no quarternary ammonium compounds are formed (by these connections); Further Q represents an unsubstituted or monosubstituted or polysubstituted (C₃-C₇)cycloalkylene wherein any substituents each individually and independently are selected from (C₁-C₆)alkyl, (C₁-C₆)alkoxyl, oxy-(C₁-C₆)alkyl, (C₂-C₆)alkenyl, (C₂-C₆)alkynyl, (C₃-C₆)cycloalkyl, (C₃-C₆)cycloakyl(C₁-C₄)alkylene, carboxyl, carboxy-(C₁-C₄)alkylene, aryl, aryl(C₁-C₄)alkylene, heterocyclyl, heterocyclyl(C₁-C₄)alkylene, nitro, cyano, halogeno (F, Cl, Br, I), hydroxyl, NR^{a(Q)}R^{b(Q)} in which R^{a(Q)} and R^{b(Q)} individually and independently from each other represents hydrogen, (C₁-C₄)alkyl or R^{a(Q)} and R^{b(Q)} together with the nitrogen atom represent piperidine, pyrrolidine, azetidine or aziridine; Further Q represents aryl wherein any substituents each individually and independently are selected from (C₁-C₆)alkyl, (C₁-C₆)alkoxyl, oxy-(C₁-C₆)alkyl, (C₂-C₆)alkenyl, (C₂-C₆)alkyl, (C₃-C₆)cycloalkyl, (C₃-C₆)cycloalkyl(C₁-C₄)alkylene, carboxyl, carboxy-(C₁-C₄)alkylene, aryl, aryl(C₁-C₄)alkylene, heterocyclyl, heterocyclyl(C₁-C₄)alkylene, nitro, cyano, halogeno (F, Cl, Br, I), hydroxyl, NR^{a(Q)}R^{b(Q)} in which R^{a(Q)} and R^{b(Q)} individually and independently from each other represents hydrogen, (C₁-C₄)alkyl or R^{a(Q)} and R^{b(Q)} together with the nitrogen atom represent piperidine, pyrrolidine, azetidine or aziridine;

R₁₆ is ethyl;

R^{c} is absent or represents an unsubstituted or monosubstituted or polysubstituted (C₁-C₄)alkylene group, (C₁-C₄)oxoalkylene group, (C₁-C₄)alkyleneoxy or oxy-(C₁-C₄)alkylene group, wherein any substituents each individually and independently are selected from (C₁-C₄)alkyl, (C₁-C₄)alkoxyl, oxy-(C₁-C₄)alkyl, (C₂-C₄)alkenyl, (C₂-C₄)alkynyl, (C₃-C₆)cycloalkyl, carboxyl, carboxy-(C₁-C₄)alkyl, aryl, heterocyclyl, nitro, cyano, halogeno (F, Cl, Br, I), hydroxyl, NR^{a(Rc)}R^{b(Rc)} in which R^{a(Rc)} and R^{b(Rc)} individually and independently from each other represents hydrogen, (C₁-C₄)alkyl or R^{a(Rc)} and R^{b(Rc)} together with the nitrogen atom represent piperidine, pyrrolidine, azetidine or aziridine; Further R^{c} represents imino (—NH—), N-substituted imino (—NR₁₉—), (C₁-C₄)alkyleneimino or N-substituted (C₁-C₄)alkyleneimino (—N(R₁₉)—((C₁-C₄)alkylene) wherein the mentioned alkylene groups are unsubstituted or monosubstituted or polysubstituted with any substituents according to above; preferably R^{c} represents imino or (C₁-C₄)alkyleneimino or an unsubstituted or monosubstituted or polysubstituted (C₁-C₄)alkylene group or (C₁-C₄)oxoalkylene group with any substituents according to above;

R₁₉ represents H or (C₁-C₄)alkyl; and

R^{d} represents (C₃-C₈)cycloalkyl, aryl or heterocyclyl, and anyone of these groups optionally substituted with one or more halogen (F, Cl, Br, I) atoms and/or one or more of the following groups, CN, NO₂, (C₁-C₆)alkyl, (C₁-C₆)alkoxy, halosubstituted (C₁-C₆)alkyl, (C₃-C₆)cycloalkyl, aryl, heterocyclyl, (C₁-C₆)alkylsulfonyl, (C₁-C₆)alkylsulfonyl, (C₁-C₆)alkylthio, (C₃-C₆)cycloalkylthio, arylsulfinyl, arylsulfonyl, arylthio, aryl(C₁-C₆)alkylthio, aryl(C₁-C₆)alkylsulfonyl, aryl(C₁-C₆)alkylsulfonyl, heterocyclyl(C₁-C₆)alkylthio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkylsulfinyl or (C₃-C₆)cycloalkyl(C₁-C₆)alkylsulfonyl.

A 4th embodiment of formula I is defined by;

R₁ represents R₆OC(O);

R₂ represents (C₁-C₆)alkyl optionally interrupted by oxygen and/or optionally substituted by one or more halogen (F, Cl, Br, I) atoms;

R₃ represents H;

R₄ represents CN or halogen (F, Cl, Br, I);

R₆ represents (C₁-C₆)alkyl optionally interrupted by oxygen, (with the proviso that any such oxygen must be at least 2 carbon atoms away from the ester-oxygen connecting the R₆ group) and/or optionally substituted by OH, aryl, cycloalkyl, heterocyclyl or one or more halogen (F, Cl, Br, I) atoms;

R₉ represents H or (C₁-C₄)alkyl;

R₁₀ represents H or (C₁-C₄)alkyl;

Q represents an unsubstituted or monosubstituted or polysubstitlited (C₁-C₄)alkylene group, wherein any substituents each individually and independently are selected from (C₁-C₆)alkyl, (C₁-C₆)alkoxyl, oxy-(C₁-C₆)alkyl, or represents an unsubstituted or monosubstituted or polysubstituted (C₃-C₇)cycloalkylene wherein any substituents each individually and independently are selected from (C₁-C₄)alkyl, (C₁-C₄)alkoxyl, oxy-(C₁-C₄)alkyl or halogeno (F, Cl, Br, I);

R^{c} is absent or represents an unsubstituted or monosubstituted (C₁-C₄)alkylene group, (C₁-C₄)alkyleneoxy or oxy-(C₁-C₄)alkylene group, wherein any substituents each individually and independently are selected from (C₁-C₄)alkyl; and

R^{d} represents aryl or heterocyclyl, and anyone of these groups optionally substituted with one or more halogen (F, Cl, Br, I) atoms and/or one or more of the following groups, CN, NO₂, (C₁-C₆)alkyl, (C₁-C₆)alkoxy, halosubstituted (C₁-C₆)alkyl.

A 5th embodiment of formula I is defined by that;
R₁ is ethoxycarbonyl;
R₂ is chosen from a group consisting methyl and trifluoromethyl;
R₃ is H;
R₄ is cyano;
R₆ is ethyl;
R₉ is H;
R₁₀ is H;
Q is a 1,3-cyclopentylene group or a methylene (—CH₂—) group;
R^{c} is absent or is methylene (—CH₂—) or ethylene (—CH₂CH₂—); and
R^{d} is chosen from a group consisting of phenyl and 5-chloro-2-thienyl.

In a 6th embodiment of formula (I), formula (I) is defined as being any compound(s) of formula (Ia)-(Ii):

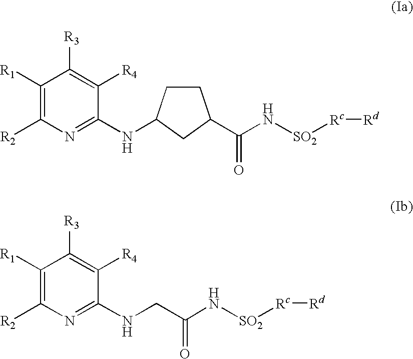

In the above Ia to Ib the various values of R (except R₉ and R₁₀ both being H) are as defined above and include the previously mentioned embodiments.

In a 7^{th} embodiment formula (I) is defined as being any compound(s) of formula (Iaa-(Ibb);

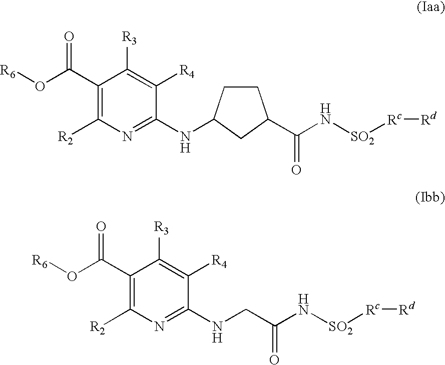

In the above Iaa to Ibb the various values of R (except R₉ and R₁₀ both being H) are as defined above and include the previously mentioned embodiments.

Examples of specific compounds according to the invention can be selected from;
ethyl 6-[(3-{[(benzylsulfonyl)amino]carbonyl}cyclopentyl)amino]-5-cyano-2-(trifluoromethyl)nicotinate
ethyl 5-cyano-6-{[3-({[(2-phenylethyl)sulfonyl]amino}carbonyl)cyclopentyl]amino}-2-(trifluoromethyl)nicotinate
ethyl 6-{[3-({[(5-chloro-2-thienyl)sulfonyl]amino}carbonyl)cyclopentyl]amino}-5-cyano-2-(trifluoromethyl)nicotinate
ethyl 6-[(2-{[(5-chloro-2-thienyl)sulfonyl]amino}-2-oxoethyl)amino]-5-cyano-2-(trifluoromethyl)nicotinate
ethyl 6-({2-[(benzylsulfonyl)amino]-2-oxoethyl}amino)-5-cyano-2-(trifluoromethyl)nicotinate
ethyl 6-({2-[(benzylsulfonyl)amino]-2-oxoethyl}amino)-5-cyano-2-(trifluoromethyl)nicotinate
ethyl 6-({3-[(benzylsulfonyl)carbamoyl]cyclopentyl}amino)-5-cyano-2-methylnicotinate;
   and pharmaceutically acceptable salts thereof.

Processes

The following processes together with the intermediates are provided as a further feature of the present invention.

Compounds of formula (I) may be prepared by the following processes a1-a4;

a1) Compounds of formula (I) in which R₁, R₂, R₃, R₄, R₉, R₁₀, Q, R^{c} and R^{d} are defined as in formula (I) above may be formed by reacting a compound of formula (II), in which R₁, R₂, R₃, R₄, Q and R₉ are defined

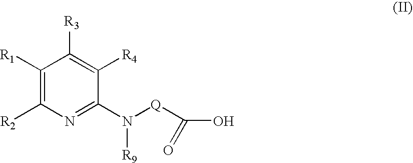

as in formula (I) above with a compound of formula (III) in which R₁₀, R^{c} and R^{d} are defined as in formula (I) above.

R₁₀—NHSO₂—R^{c}—R^{d} (III)

The reaction is generally carried out in an inert organic solvent such as dichloromethane at ambient temperature. The reaction may be carried out using standard conditions or in the presence of TBTU, EDCI or the combination of EDCI and HOBT. Optionally, the reaction may be carried out in the presence of an organic base such as triethylamine or DIPEA.

a2) Compounds of formula (I) may also be prepared by reacting a compound of formula (IV) in which R₁, R₂, R₃ and R⁴ are defined as in formula (I) above and L is a suitable leaving group, such as chloro, bromo, iodo, fluoro, triflate (OTf) or tosylate (OTs),

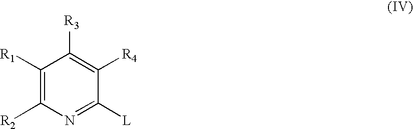

with a compound of the general formula (V) in which R₉, R¹⁰, Q, R^{c} and R^{d} are defined as in formula (I) above.

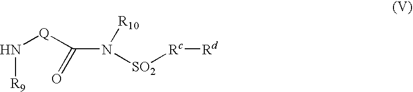

The reaction is generally carried out in an inert solvent such as DMA. Optionally, the reaction may be carried out in the presence of an organic base such as triethylamine or DIPEA.

The reaction is generally carried out at elevated temperatures using standard equipment or in a single-node microwave oven.

For some compounds, it is advantageous to carry out the reaction in ethanol in the presence of an organic base such as triethylamine.

a3) Compounds of formula (I) where R₁ represents R₆OC(O) and R₂, R₃, R₄, R₆, R₉, R₁₀, Q, R^{c} and R^{d} are defined as in formula (I) above, can be transesterified using standard procedures or by reacting with R_{6′}—O⁻Li⁺ reagent, to become another compound of the general formula (I) wherein R₁ becomes R_{6′}OC(O).

a4) The compounds of formula (I) in which R₁ is R₆OC(O) and R₃, R₄, R₆, R₉, R₁₀, Q, R^{c} and R_{d} are as defined in formula (I) above, R₂ is (C₁-C₁₂)alkoxy defined as in formula (I) above may be prepared by reacting a compound of formula (VI)

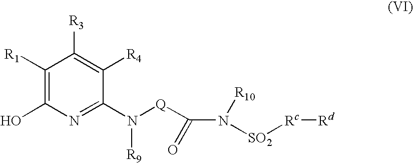

in which R₁ is R₆OC(O) and R₃, R₄, R₆, R₉, R₁₀, Q, R^{c} and R^{d} are as defined in formula (I) above with a compound of formula (VII)

L-R_{2′} (VII)

in which R₂ is (C₁-C₁₂)alkyl defined as in formula (I) and L is a leaving group such as chloro, bromo, iodo, triflate (OTf) or tosylate (OTs).

The reaction is carried out in an inert organic solvent such as DMA, THF or CH₃CN.

The reaction may be carried out using standard conditions or in the presence of a suitable base such as sodium hydride, DIPEA or potassium carbonate.

The reaction may be carried out at ambient temperature or at elevated temperatures using standard equipment or a single node microwave oven.

The intermediates referred to above may be prepared by, for example, the methods/processes outlined below.

b) The compounds of formula (II) in which R₁, R₂, R₃, R₄, R₉, and Q are defined as in formula (I) above may be prepared by reacting a compound of formula (IV),

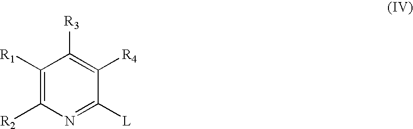

in which R₁, R₂, R₃ and R^{a} are defined as in formula (I) above and L is a suitable leaving group (such as fluoro, chloro, bromo, iodo, triflate (OTf) or tosylate (OTs)), with a compound of the general formula (VIII),

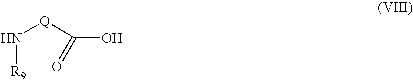

in which R₉ and Q are defined as in formula (I) above.

The reaction is generally carried out at elevated temperatures using standard equipment or in a single-node microwave oven. The reaction can be carried out in an inert solvent such as ethanol, DMA or a mixture of solvents such as ethanowater. Optionally the reaction may be carried out in the presence of an organic base such as TEA or DIPEA.

d) Synthesis of compounds of the general formula (IX),

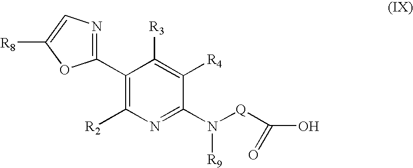

in which R₂, R₃, R₄, R₅, R₉ and Q are defined as in formula (I) above comprises the below steps. (d1-d5)

d1) Reacting the corresponding compounds of the general formula (VI) which is defined as above with a compound of the general formula (X)

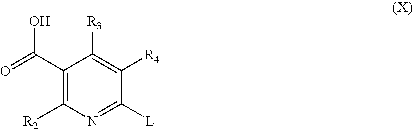

in which R₂, R₃ and R₄ are defined as in formula (I) above, and L is a suitable leaving group, such as chloro, bromo, iodo, triflate (OTf) or tosylate (OTs), to give a compound of formula (XI).

The reactions are carried out at elevated temperatures using standard equipment or a single-node microwave oven. Optionally the reaction may be carried out in the prescence of an organic base such as TEA or DIPEA.

d2) The compounds of formula (XI) can then be reacted

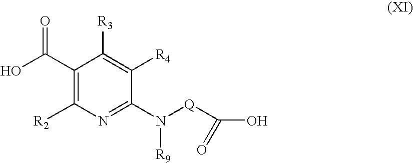

with a compound of the general formula (XII),

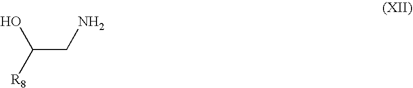

in which % is defined as in formula (I) above, to give compounds of the general formula (XIII). The reactions are carried out using standard conditions or in the prescence of EDCI or the combination of EDCI and HOBT. Optionally the reaction may be carried out in the prescence of an organic base such as TEA or DIPEA.

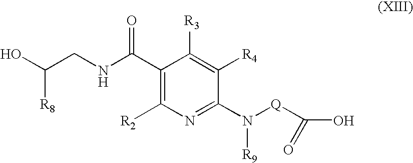

d3) This compound (XIII) can then be transformed to a compound of the general formula (XIV)

d4) The preparation of compounds with the general formula (XIV),

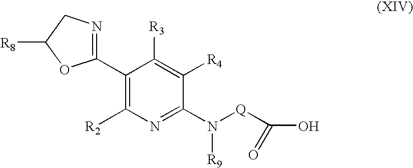

in which R₂, R₃, R₄, B, R₈, R₉ and Q are defined as in formula (I) above and using known methods or a known reagent such as methanesulfonyl chloride. Optionally the reaction may be carried out in the prescence of an organic base such as TEA.

d5) a compound of the general formula (IX) as defined above can be made by oxidizing the corresponding compound of the general formula (XIV) using a known oxidation reagent such as DDQ.

e) The preparation of compounds of the general formula (IX) also comprises the steps (e1-e4) below;
e1) Reacting a compound the general formula (XV),

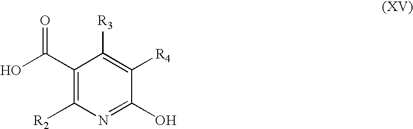

in which R₂, R₃ and R₄ are defined as in formula (I) above, with a compound of the general formula (XVI), in which % is defined as in formula (I) above,

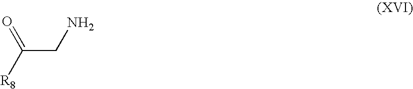

using standard conditions or in the prescence of EDCI or the combination of EDCI and HOBT. Optionally the reaction may be carried out in the prescence of an organic base such as TEA. This reaction gives a compound of the general formula (XVII).

e2) The compound of the general formula (XVII) obtained

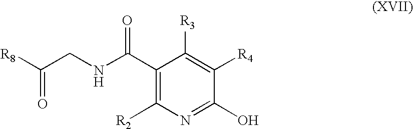

can then be transformed to a compound of the general formula (XVIII), in which R₂, R₃, R₄ and R₈ are defined as in formula (I) above, using known techniques or using a known reagent such as POCl₃.

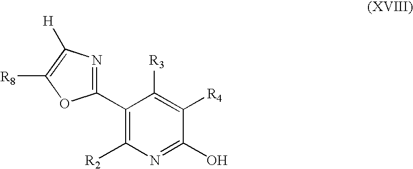

e3) A compound of the general formula (XVIII) can then be transformed to a compound of the general formula (XIX),

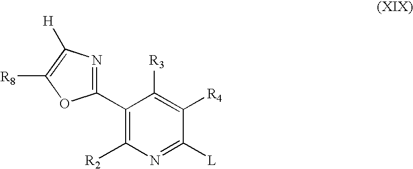

in which R₂, R₃, R₄, R₈ are defined as in formula (I) above and L is a sufficient leaving group, such as chloro, bromo, iodo, triflate (OTf) or tosylate (OTs), using a known techniques or a reagent such as oxalyl chloride or thionyl chloride.

e4) The compound of formula (XIX) can then be reacted with a compound of the general formula (VII), which is defined as above, to give a compound of the general formula (IX), defined as above. The reactions are carried out at elevated temperatures using standard equipment or a single-node microwave oven. Optionally the reactions may be carried out in the prescence of an organic base such as TEA or DIPEA.

Compounds of the general formula (II), in which R₁ is R₇C(O) and R₂, R₃, R₄, R₇, R₉ and Q are defined as in formula (I) above, comprises the following steps (f1-f2):

f1) Reacting a compound of the general formula (XI), described above, with N,O-dimethylhydroxylamine. The reaction can be performed using known reagents like CDI, EDCI or the combination of EDCI and HOBT to give a compound of the general formula (XX).

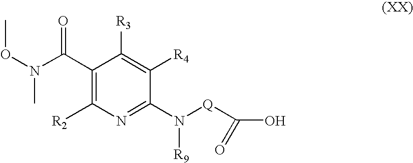

f2) Reacting compounds of the general formula (XX), defined as above, with a reagent of the general formula R₇-MgX, in which R₇ is defined as in formula (I) above and X is a halogen, or a reagent of the formula R₇-M, in which M is a metal exemplified by Zn and Li.

g) Compounds of the general formula (V) in which R₉, R₁₀, Q, R^{c} and R^{d} are defined as in formula (I) above may be formed by reacting a compound of formula (VIII) with a compound of formula (II). The reaction is generally carried out in an inert organic solvent such as dichloromethane at ambient temperature. The reaction may be carried out using standard conditions or in the presence of EDCI or the combination of EDCI and HOBT. Optionally, the reaction may be carried out in the presence of an organic base such as triethylamine or DIPEA.

(h) Compounds of the general formula (IV) which are defined as above can be formed by reacting a compound of formula (XXI) using standard conditions or with a chlorinating reagent such as thionyl chloride or POCl₃. Advantageously dimethylformamide may be used. The reaction may be performed in an inert solvent. Advantageously the inert solvent is toluene.

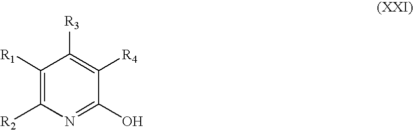

The preparation of compounds of the general formula (XXII) which is defined as above comprises the steps (i1-i3) below;

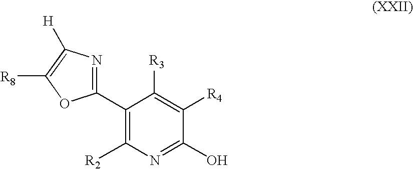

i1) Reacting a compound of the general formula (XXIII)

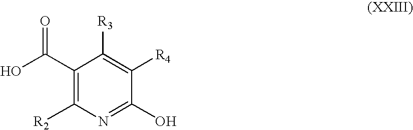

with a compound of the general formula (XII) to give a compound of the formula (XXIV). The reaction is generally carried out in DCM at ambient temperature. The reaction may be carried out using standard conditions or in the presence of EDCI or the combination of EDCI and HOBT. Optionally the reaction may be carried out in the prescence of an organic base such as TEA or DIPEA.

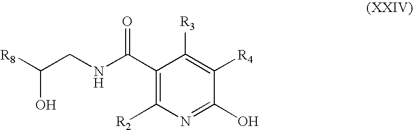

i2) The compound of formula (XXIV) can be transformed to a compound (XXV) using standard conditions or an oxidizing agent such as the mixture of oxalylchloride and DMSO.

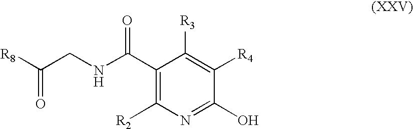

i3) The compound of formula (XXV) can then be transformed into a compound of the general formula (XXII), using standard conditions or in the presence of (ethoxycarbonylsulfamoyl)triethylammonium hydroxide (Burgess reagent). The reaction is generally performed in an inert solvent such as THF. The reaction is carried out at elevated temperatures using standard equipment or a single-node microwave oven.

Compounds of the general formula (III) can be formed by reacting the corresponding sulfonyl chloride using known methods with ammonia or R₁₀NH₂ in an inert solvent such as methanol, THF or DCM.

j) Preparation of compounds of the general formula (XXIII) which is defined as above except for R₃ which is hydrogen, comprises the following steps (j1-j3);

j1) Reacting a compound of the formula (XXVI), in which 12 and R₆ are defined as in formula (I) above with dimethoxy-N,N-dimethylmethaneamine to form a

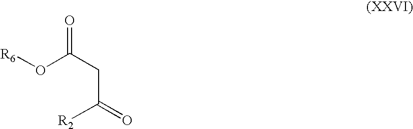

compound of formula (XXVII).

j2) This compound (XXVII) can then be reacted further with a compound of the

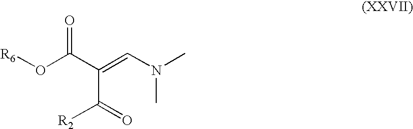

general formula R₄CH₂C(O)NH₂, in which R₄ is defined as in formula (I) above to give a compound of the general formula (XXVIII). The reaction is generally performed in an inert solvent such as ethanol, optionally in the presence of a strong base such as sodium ethoxide.

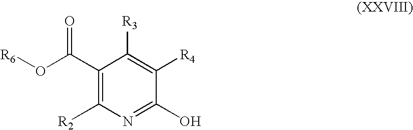

j3) A compound of the general formula (XXVIIII) can then be transformed to a compound of the general formula (XXIII). The reaction is generally performed in a protic solvent such as water together with a co-solvent such as THF or methanol. The reaction can be performed using standard reagents or in the presence of LiOH, NaOH or KOH.

(k) The formation of a compound of the general formula (IX), which is defined as above can be made the below synthesis;

k1) A compound of the general formula (XXIX) where R₈ is defined as in formula (I) above can be

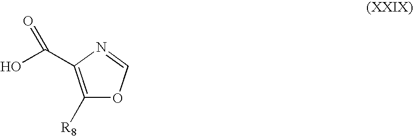

transformed in to a compound of the formula (XXX)

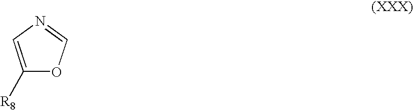

using standard conditions or using Cu(II)O and quinoline.

k2) The compound of the general formula (XXX) can be reacted with a compound of the general formula (XXXI) in

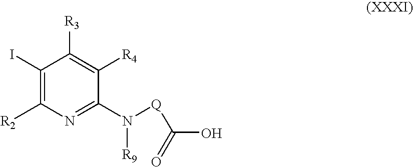

which R₂, R₃, R₄, R₉ and Q are defined as for formula (I) to give compounds of the general formula (IX). The reaction is generally performed in an inert solvent such as THF under inert atmosphere. The reaction can be performed using standard conditions or in the presence of AlkylLi such as BuLi followed by treatment with ZnCh and Pd(PPh₃)₄ (preferably a catalytic amount).

l) Compounds of the general formula (VI) defined above can be prepared by the following steps l1-l2 below

l1) Reacting a compound of the general formula (XXXII)

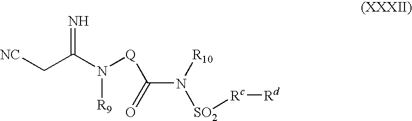

where R₉, R₁₀, Q, R^{c} and R^{d} are as defined in formula (I) above with a compound of formula (XXIII)

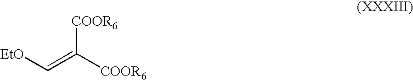

The reaction is generally carried out in an inert organic solvent such as EtOH or DMSO.

The reaction is carried out at ambient temperature or at elevated temperatures using standard equipment or a single node microwave oven.

l2) Compounds of the general formula (XXXI) defined above can be prepared by reacting a compound of the general formula (V) as defined above with a compound of formula (XXIV)

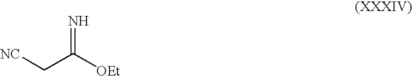

using essentially the same procedure as described in [Macconi, A et. Al., J. Heterocyclic chemistry, 26, p. 1859 (1989)].

The preparation of compounds of the formula (II) comprises the below processes. (m1-m3)

m1) A compound of the formula LR^{c}R^{d} wherein L is a suitable leaving group, such as chloro, bromo, iodo could be transformed to the corresponding compound (III) using a sequence of reactions using first SMOPS* (*Baskin and Wang. Tetrahedron Letters, 2002, 43, 8479-83. See esp. page 8480, left hand column.) followed by hydrolysis using a base like NaOMe in an inert solvent like DMSO at room temperature. Followed by treatment by NH₂OSO₃H and NaOAc to give a compound of formula (III) in which R₁₀ is H.

m2) A compound of the formula LSO₂R^{c}R^{d} wherein L is a suitable leaving group, such as chloro, bromo, iodo could be reacted with ammonium hydroxide or H₂NR₁₀ in an inert solvent such as DCM to give a compound of formula (III).

m3) A compound of the formula LR^{c}R^{d} wherein L is a suitable leaving group, such as chloro, bromo, iodo could be transformed to the corresponding compound (III) using a sequence of reactions first NaSO₃, followed by a using a reagent such as PCl₆, POCl₃ or SOCl₂, followed by ammonium hydroxide or H₂NR₁₀ to give a compound of formula (III).

At any stage in the synthesis of amine substituted pyridines, a halogen substituent in the 2, 4 or 6 position of the pyridine can be substituted with azide using known techniques. The azide can be reduced to the corresponding amine. These amines can subsequently be alkylated or acylated using known methods or with an alkylhalide or alkylhalide, respectively.

Persons skilled in the art will appreciate that an acid can be transformed to the corresponding activated ester such as an acid chloride, followed by reaction with a thiol, R₁₆SH to give thioesters, R₁₆SC(O)

Persons skilled in the art will appreciate that an acid can be transformed to the corresponding activated ester such as an acid chloride, followed by reaction with a alcohol, R₆OH to give esters, R₆OC(O).

Persons skilled in the art will appreciate that a compound of formula (III) could be alkylated at the carbon atom in the alpha position to the sulfonamide using an alkylhalide. Preferably under basic conditions using a strong base such as sodium hydride.

Persons skilled in the art will appreciate that a nitrogen substituent at the 3 position of a pyridine could be replaced by a thioether chain, R₁₇S—, using known techniques or R₁₇SSR₁₇ and tert-Butylnitrile.

Persons skilled in the art will appreciate that a thioketone or thioamide could be made from the corresponding ketone or amide respectively, using known techniques or using Lawessons reagent.

The compounds of the invention may be isolated from their reaction mixtures using conventional techniques.

Persons skilled in the art will appreciate that, in order to obtain compounds of the invention in an alternative and in some occasions, more convenient manner, the individual process steps mentioned hereinbefore may be performed in different order, and/or the individual reactions may be performed at different stage in the overall route (i.e. chemical transformations may be performed upon different intermediates to those associated hereinbefore with a particular reaction).

It will be appreciated that by those skilled in the art that the processes described above and hereinafter the functional groups of intermediate compounds may need to be protected by protecting groups.

Functional groups that it is desirable to protect include hydroxy, amino and carboxylic acid. Suitable protecting groups for hydroxy include optionally substituted and/or unsaturated alkyl groups (e.g. methyl, allyl, benzyl or tert-butyl), trialkyl silyl or diarylalkylsilyl groups (e.g. t-butyldimethylsilyl, t-butyldiphenylsilyl or trimethylsilyl) and tetrahydropyranyl. Suitable protecting groups for carboxylic acids include (C₁-C₆)alkyl or benzyl esters. Suitable protecting groups for amino include t-butyloxycarbonyl, benzyloxycarbonyl, 2-(trimethylsilyl)ethoxymethyl or 2-trimethylsilylethoxycarbonyl (Teoc).

The protection and deprotection of functional groups may take place before or after any reaction in the above mentioned processes.

Persons skilled in the art will appreciate that, in order to obtain compounds of the invention in an alternative, and on some occasions, more convenient, manner, the individual process steps mentioned hereinbefore may be performed in different order, and/or the individual reactions may be performed at a different stage in the overall route (i.e. substituents may be added to and/or chemical transformations performed upon, different intermediates to those mentioned hereinbefore in conjunction with a particular reaction). This may negate, or render necessary, the need for protecting groups.

Persons skilled in the art will appreciate that starting materials for any of the above processes can in some cases be commercially available.

Persons skilled in the art will appreciate that processes for some starting materials above could be found in the general common knowledge.

The type of chemistry involved will dictate the need for protecting groups as well as sequence for accomplishing the synthesis.

The use of protecting groups is fully described in “Protective groups in Organic Chemistry”, edited by J W F McOmie, Plenum Press (1973), and “Protective Groups in Organic Synthesis”, 3^{rd} edition, T. W.-Greene & P. G. M Wutz, Wiley-Interscience (1999).

Protected derivatives of the invention may be converted chemically to compounds of the invention using standard deprotection techniques (e.g. under alkaline or acidic conditions). The skilled person will also appreciate that certain compounds of Formula (II))-(XXXIV) may also be referred to as being “protected derivatives”

Compounds of the invention may also contain one or more asymmetric carbon atoms and may therefore exhibit optical and/or diastereoisomerism. Diastereoisomers may be separated using conventional techniques, e.g. chromatography or crystallization. The various stereoisomers may be isolated by separation of a racemic or other mixture of the compounds using conventional, e.g. HPLC techniques. Alternatively the desired optical isomers may be made by reaction of the appropriate optically active starting materials under conditions which will not cause racemisation or epimerization, or by derivatisation, for example with a homochiral acid followed by separation of the diasteromeric derivatives by conventional means (e.g. HPLC, chromatography over silica or crystallization). Stereo centers may also be introduced by asymmetric synthesis, (e.g. metalloorganic reactions using chiral ligands). All stereoisomers are included within the scope of the invention.

All novel intermediates form a further aspect of the invention.

Salts of the compounds of formula (I) may be formed by reacting the free acid, or a salt thereof, or the free base, or a salt or a derivative thereof, with one or more equivalents of the appropriate base (for example ammonium hydroxide optionally substituted by C₁-C₆-alkyl or an alkali metal or alkaline earth metal hydroxide) or acid (for example a hydrohalic (especially HCl), sulphuric, oxalic or phosphoric acid). The reaction may be carried out in a solvent or medium in which the salt is insoluble or in a solvent in which the salt is soluble, e.g. water, ethanol, tetrahydrofuran or diethyl ether, which may be removed in vacuo, or by freeze drying. The reaction may also carried out on an ion exchange resin. The nontoxic physiologically acceptable salts are preferred, although other salts may be useful, e.g. in isolating or purifying the product.

### Pharmacological Data

Functional inhibition of the P2Y₁₂ receptor can be measured by in vitro assays using cell membranes from P2Y₁₂ transfected CHO-cells, the methodology is indicated below.

Functional inhibition of 2-Me-S-ADP induced P2Y₁₂ signalling: 5 μg of membranes were diluted in 200 μl of 200 mM NaCl, 1 mM MgCl₂, 50 mM HEPES (pH 7.4), 0.01% BSA, 30 μg/ml saponin and 10 μM GDP. To this was added an EC₈₀ concentration of agonist (2-methyl-thio-adenosine diphosphate), the required concentration of test compound and 0.1 μCi ³⁵S-GTPγS. The reaction was allowed to proceed at 30° C. for 45 min. Samples were then transferred on to GF/B filters using a cell harvester and washed is with wash buffer (50 mM Tris (pH 7.4), 5 mM MgCl₂, 50 mM NaCl). Filters were then covered with scintilant and counted for the amount of ³⁵S-GTPγS retained by the filter. Maximum activity was that determined in the presence of the agonist and minimum activity in the absence of the agonist following subtraction of the value determined for non-specific activity. The effect of compounds at various concentrations was plotted according to the equation

y=A+((B−A)/(1+((C/x)̂D)))

and IC₅₀ estimated where
A is the bottom plateau of the curve i.e. the final minimum y value
B is the top of the plateau of the curve i.e. the final maximum y value
C is the x value at the middle of the curve. This represents the log EC₅₀ value when A+B=100
D is the slope factor.
x is the original known x values.
Y is the original known y values.

Most of the compounds of the invention have an activity, when tested in the functional inhibition of 2-Me-S-ADP induced P2Y₁₂ signalling assay described, at a concentration of around 4 μM or below.

For example the compounds described in Examples 3 and 6 gave the following test result in the functional inhibition of 2-Me-S-ADP induced P2Y₁₂ signalling assay described.

IC₅₀(μM)

| | |
|---|---|
| Example 3 | 0.81 |
| Example 6 | 0.24 |

The compounds of the invention act as P2Y₁₂ receptor antagonists and are therefore useful in therapy. Thus, according to a further aspect of the invention there is provided a compound of formula (I), or a pharmaceutically acceptable salt thereof, for use in therapy.

In a further aspect there is provided the use of a compound of formula (I), or a pharmaceutically acceptable salt thereof, for the manufacture of a medicament for treatment of a platelet aggregation disorder. In another aspect of the invention there is provided the use of a compound of formula (I), or a pharmaceutically acceptable salt thereof, for the manufacture of a medicament for the inhibition of the P2Y₁₂ receptor.

The compounds are useful in therapy, especially adjunctive therapy, particularly they are indicated for use as: inhibitors of platelet activation, aggregation and degranulation, promoters of platelet disaggregation, anti-thrombotic agents or in the treatment or prophylaxis of unstable angina, coronary angioplasty (PTCA), myocardial infarction, perithrombolysis, primary arterial thrombotic complications of atherosclerosis such as thrombotic or embolic stroke, transient ischaemic attacks, peripheral vascular disease, myocardial infarction with or without thrombolysis, arterial complications due to interventions in atherosclerotic disease such as angioplasty, endarterectomy, stent placement, coronary and other vascular graft surgery, thrombotic complications of surgical or mechanical damage such as tissue salvage following accidental or surgical trauma, reconstructive surgery including skin and muscle flaps, conditions with a diffuse thrombotic/platelet consumption component such as disseminated intravascular coagulation, thrombotic thrombocytopenic purpura, hemolytic uraemic syndrome, thrombotic complications of septicaemia, adult respiratory distress syndrome, anti-phospholipid syndrome, heparin-induced thrombocytopenia and pre-eclampsia/eclampsia, or venous thrombosis such as deep vein thrombosis, venoocclusive disease, haematological conditions such as myeloproliferative disease, including thrombocythemia, sickle cell disease; or in the prevention of mechanically-induced platelet activation in vivo, such as cardio-pulmonary bypass and extracorporeal membrane oxygenation (prevention of microthromboembolism), mechanically-induced platelet activation in vitro, such as use in the preservation of blood products, e.g. platelet concentrates, or shunt occlusion such as in renal dialysis and plasmapheresis, thrombosis secondary to vascular damage/inflammation such as vasculitis, arteritis, glomerulonephritis, inflammatory bowel disease and organ graft rejection, conditions such as migraine, Raynaud's phenomenon, conditions in which platelets can contribute to the underlying inflammatory disease process in the vascular wall such as atheromatous plaque formation/progression, stenosis/restenosis and in other inflammatory conditions such as asthma, in which platelets and platelet-derived factors are implicated in the immunological disease process.

According to the invention there is further provided the use of a compound according to the invention in the manufacture of a medicament for the treatment of the above disorders. In particular the compounds of the invention are useful for treating myocardial infarction, thrombotic stroke, transient ischaemic attacks, peripheral vascular disease and angina, especially unstable angina. The invention also provides a method of treatment of the above disorders which comprises administering to a patient suffering from such a disorder a therapeutically effective amount of a compound according to the invention.

In a further aspect the invention provides a pharmaceutical composition comprising a compound of formula (I), or a pharmaceutically acceptable salt thereof, together with a pharmaceutically acceptable diluent, adjuvant and/or carrier.

The compounds may be administered topically, e.g. to the lung and/or the airways, in the form of solutions, suspensions, HFA aerosols and dry powder formulations; or systemically, e.g. by oral administration in the form of tablets, pills, capsules, syrups, powders or granules, or by parenteral administration in the form of sterile parenteral solutions or suspensions, by subcutaneous administration, or by rectal administration in the form of suppositories or transdermally.

The compounds of the invention may be administered on their own or as a pharmaceutical composition comprising the compound of the invention in combination with a pharmaceutically acceptable diluent, adjuvant or carrier. Particularly preferred are compositions not containing material capable of causing an adverse, e.g. an allergic, reaction.

Dry powder formulations and pressurised HFA aerosols of the compounds of the invention may be administered by oral or nasal inhalation. For inhalation the compound is desirably finely divided. The compounds of the invention may also be administered by means of a dry powder inhaler. The inhaler may be a single or a multi dose inhaler, and may be a breath actuated dry powder inhaler.

One possibility is to mix the finely divided compound with a carrier substance, e.g. a mono-, di- or polysaccharide, a sugar alcohol or another polyol. Suitable carriers include sugars and starch. Alternatively the finely divided compound may be coated by another substance. The powder mixture may also be dispensed into hard gelatine capsules, each containing the desired dose of the active compound.

Another possibility is to process the finely divided powder into spheres, which break up during the inhalation procedure. This spheronized powder may be filled into the drug reservoir of a multidose inhaler, e.g. that known as the Turbuhaler in which a dosing unit meters the desired dose which is then inhaled by the patient. With this system the active compound with or without a carrier substance is delivered to the patient.

The pharmaceutical composition comprising the compound of the invention may conveniently be tablets, pills, capsules, syrups, powders or granules for oral administration; sterile parenteral or subcutaneous solutions, suspensions for parenteral administration or suppositories for rectal administration.

For oral administration the active compound may be admixed with an adjuvant or a carrier, e.g. lactose, saccharose, sorbitol, mannitol, starches such as potato starch, corn starch or amylopectin, cellulose derivatives, a binder such as gelatine or polyvinylpyrrolidone, and a lubricant such as magnesium stearate, calcium stearate, polyethylene glycol, waxes, paraffin, and the like, and then compressed into tablets. If coated tablets are required, the cores, prepared as described above, may be coated with a concentrated sugar solution which may contain e.g. gum arabic, gelatine, talcum, titanium dioxide, and the like. Alternatively, the tablet may be coated with a suitable polymer dissolved either in a readily volatile organic solvent or an aqueous solvent.

For the preparation of soft gelatine capsules, the compound may be admixed with e.g. a vegetable oil or polyethylene glycol. Hard gelatine capsules may contain granules of the compound using either the above mentioned excipients for tablets, e.g. lactose, saccharose, sorbitol, mannitol, starches, cellulose derivatives or gelatine. Also liquid or semisolid formulations of the drug may be filled into hard gelatine capsules.

Liquid preparations for oral application may be in the form of syrups or suspensions, for example solutions containing the compound, the balance being sugar and a mixture of ethanol, water, glycerol and propylene glycol. Optionally such liquid preparations may contain colouring agents, flavouring agents, saccharine and carboxymethylcellulose as a thickening agent or other excipients known to those skilled in art.

The invention will be further illustrated with the following non-limiting examples:

### EXAMPLES

### General Experimental Procedure

Mass spectra was recorded on a Finnigan LCQ Duo ion trap mass spectrometer equipped with an electrospray interface (LC-MS) or LC-MS system consisting of a Waters ZQ using a LC-Agilent 1100 LC system. ¹H NMR measurements were performed on a Varian Mercury VX 400 spectrometer, operating at a 1H frequency of 400 and Varian UNITY plus 400, 500 and 600 spectrometers, operating at 1H frequencies of 400, 500 and 600, respectively. Chemical shifts are given in ppm with the solvent as internal standard. Protones on heteroatoms such as NH and OH protons are only reported when detected in NMR and can therefore be missing. HPLC separations were performed on a Waters YMC-ODS AQS-3 120 Angstrom 3×500 mm or on a Waters Delta Prep Systems using Kromasil C8, 10 μm columns.

The purification system and LC-MS system used in Method A below was Waters Fraction Lynx II Purification System: Column: Sunfire Prep C18, 5 μm OBD, 19×100 mm column. Gradient 5-95% CH₃CN in 0.1 mM HCOOH (pH=3). MS triggered fraction collection was used. Mass spectra were recorded on either Micromass ZQ single quadropole or a Micromass quattro micro, both equipped with a pneumatically assisted electrospray interface.

Reactions performed in a microwave reactor were performed in a Personal Chemistry Smith Creator, Smith synthesizer or an Emrys Optimizer.

### List of Used Abbreviations:

| Abbreviation | Explanation |
|---|---|
| aq | Aqueous |
| br | Broad |
| Brine | A saturated solution of sodium chloride in water |
| BSA | Bovine Serum Albumine |
| CDI | Carbonyldiimidazole |
| d | Doublet |
| DCM | Dichloromethane |
| DDQ | 2,3-Dichloro-5,6-dicyano-1,4-benzoquinone |
| DIPEA | N,N-Diisopropylethylamine |
| DMA | N,N-Dimethylacetamide |
| DMF | N,N-dimethylformamide |
| DMSO | Dimethylsulphoxide |
| EDCI | N-[3-(dimethylamino)propyl]-N′-ethylcarbodiimide |
| | hydrochloride |
| EtOAc | Ethyl acetate |
| EtOH | Ethanol |
| HEPES | [4-(2-hydroxyethyl)-1-piperazineethanesulfonic |
| | acid |
| HFA | Hydrofluoroalkanes |
| HOBT | 1-Hydroxybenzotriazole |
| HPLC | High-performance liquid chromatography |
| Hz | Hertz |
| J | Coupling constant |
| LC | Liquid chromatography |
| m | Multiplet |
| MHz | Megahertz |
| mL | Millilitre |
| MS | Mass spectra |
| NCS | N-chlorosuccinimide |
| NMR | Nuclear magnetic resonance |
| OAc | acetate |
| q | Quartet |
| r.t | Room temperature |
| s | Singlet |
| t | triplet |
| TB | Tyrodes Buffer |
| TBTU | N-[(1H-1,2,3-benzotriazol-1- |
| | yloxy)(dimethylamino)methylene]-N- |
| | methylmethanaminium tetrafluoroborate |
| TEA | Triethylamine |
| Tf | trifluoromethylsulfonyl |
| THF | Tetrahydrofurane |
| TMEDA | N,N,N′,N′-tetramethylethylendiamine |
| Ts | p-toluenesulfonyl |

### Sulphone Amides

### Synthesis of Sulfone Amides

The synthesis of the sulfonamides used in the examples below was made with one of the three methods described below:

i) By reacting the corresponding sulfonyl chloride with ammonia in THF or MeOH or by treatment with ammonium hydroxide in methylene chloride. The sulfonamides obtained was used without further purification.
ii) By essentially following the procedure described by Seto, T. et. al. in J. Organic Chemistry, Vol 68, No 10 (2003), pp. 4123-4125.
or
iii) By essentially following the procedure described by Wang, Z et. al. in Tetrahedron Letters, Vol 43 (2002), pp 8479-8483.

### Synthesis of Examples

### Method A: Exemplified by the Procedure from Example 2

DIPEA (0.17 mL, 1.0 mmol) was added to a solution of ethyl 6-chloro-5-cyano-2-(trifluoromethyl)nicotinate (74 mg, 0.2 mmol) and TBTU (77 mg, 0.24 mmol) in DCM (7 mL) and the mixture was stirred for 20 min at r.t before 1-phenylethanesulfonamide (44.5 mg, 0.24 mmol) dissolved in DCM (1 mL) was added and the reaction was left over night. The reaction mixture was washed with 1% KHSO₄, the aqueous phase was extracted with DCM and the combined organic phases passed through a phase separator and evaporated in vacuum centrifuge. The crude product obtained was purified by HPLC (See General experimental procedure) to give ethyl 5-cyano-6-{[3-({[(2-phenylethyl)sulfonyl]amino}carbonyl)cyclopentyl]amino}-2-(trifluoromethyl)nicotinate. Yield: 68 mg (63%).

### Example 1

### Ethyl 6-[(3-{[(benzylsulfonyl)amino]carbonyl}cyclopentyl)amino]-5-cyano-2-(trifluoromethyl)nicotinate

### (a) Ethyl 6-chloro-5-cyano-2-(trifluoromethyl)nicotinate

Oxalylchloride (12.20 g, 96.1 mmol) and DMF (0.744 mL) were added to a solution of ethyl 5-cyano-6-oxo-2-(trifluoromethyl)-1,6-dihydropyridine-3-carboxylate (5 g, 19.22 mmol) (prepared essentially according to the method described in Mosti, L et al, Farmaco, Vol 47, No 4, 1992, pp 427-437) and the reaction was heated to 50° C. over night. The reaction was evaporated and the crude was dissolved in EtOAc and water. The phases was separated and the organic phase was washed with Brine and NaHCO₃ (aq,sat). The aqueous phase was extracted with EtOAc (3 times) and the combined organic phase was dried (Na2CO3), filtered and concentrated to give ethyl 6-chloro-5-cyano-2-(trifluoromethyl)nicotinate as a brown solid which was used without further purification. Yield: 5.206 g (95%).

¹H NMR (400 MHz, DMSO-d₆): δ 1.31 (t, J=7.2 Hz, 3H), 4.38 (q, J=6.9 Hz, 2H), 9.07 (s, 1H).

### (b) 3-{[3-Cyano-5-(ethoxycarbonyl)-6-(trifluoromethyl)pyridin-2-yl]amino}cyclopentanecarboxylic acid

TEA (0.5 mL, 6 mmol) was added to a solution of ethyl 6-chloro-5-cyano-2-(trifluoromethyl)nicotinate (341 mg, 1.2 mmol) and 3-aminocyclopentanecarboxylic acid (156 mg, 1.7 mmol) in EtOH (4.5 mL). The mixture was heated in a microwave reactor at 120° C. for 20 min. As starting material was still left more 3-aminocyclopentanecarboxylic acid (75 mg, 0.58 mmol) and TEA (0.3 mL) were added and the mixture was heated in a microwave reactor at 120° C. for another 20 min. The solution was evaporated and the solid diluted with DCM and washed with 1% KHSO₄. The combined aqueous phases were extracted with DCM and the combined organic phases filtered through a phase separator and concentrated. The crude product was purified through prepHPLC [Kromasil C8, Gradient 0 to 100% (0.2% HOAc in 5% CH₃CN/CH₃CN)] to afford a brown solid, 3-{[3-cyano-5-(ethoxycarbonyl)-6-(trifluoromethyl)pyridin-2-yl]amino}cyclopentanecarboxylic acid. Yield: 236 mg (52%)

¹H NMR (400 MHz, CDCl₃): δ 1.35 (3H, t, J=7.3 Hz), 1.91-1.77 (1H, m), 2.22-1.96 (3H, m), 2.35-2.22 (1H, m), 3.13-3.01 (1H, m), 4.34 (2H, q, J=7.2 Hz), 4.76-4.61 (1H, m), 6.70-6.58 (1H, m), 8.20 (1H, s).

MS ^{m}/_{z}: 372 (M+1).

### (c) Ethyl 6-[(3-{[(benzylsulfonyl)amino]carbonyl}cyclopentyl)amino]-5-cyano-2-(trifluoromethyl)nicotinate

DIPEA (0.17 mL, 1.0 mmol) was added to a solution of 3-{[3-cyano-5-(ethoxycarbonyl)-6-(trifluoromethyl)pyridin-2-yl]amino}cyclopentanecarboxylic acid (74.2 mg, 0.2 mmol) and TBTU (77 mg, 0.24 mmol) in DCM (7 mL) and the mixture was stirred for 20 min at r.t before 1-phenylmethanesulfonamide (41 mg, 0.24 mmol) dissolved in DCM (1 mL) was added and the reaction was left over night. The reaction mixture was washed with 1% KHSO₄, the aqueous phase was extracted with DCM and the combined organic phases passed through a phase separator and evaporated in vacuum centrifuge. The crude product obtained was purified by prepHPLC [Kromasil C8, Product loaded at pH=7 (5% CH₃CN in 0.1 M NH₄OAc(aq) and then a gradient, 20 to 100% (CH₃CN/5% CH₃CN in 0.2% AcOH)] to give a white solid, ethyl 6-[(3-{[(benzylsulfonyl)amino]carbonyl}cyclopentyl)amino]-5-cyano-2-(trifluoromethyl)nicotinate ethyl. Yield: 97 mg (88%).

1H NMR (400 MHz, CDCl₃): δ 8.55 (1H, s), 8.22 (1H, s), 7.41-7.27 (5H, m), 6.67-6.57 (1H, m), 4.72-4.57 (m), 4.33 (2H, q, J=7.7 Hz), 2.82-2.71 (1H, m), 2.28-2.16 (1H, m), 2.09-1.75 (m), 1.35 (3H, q, J=5.0 Hz).

MS ^{m}/_{z}: 525 (M+1).

### Example 2

### Ethyl 5-cyano-6-{[3-({[(2-phenylethyl)sulfonyl]amino}carbonyl)cyclopentyl]amino}-2-(trifluoromethyl)nicotinate

Prepared according to Method A from 3-{[3-cyano-5-(ethoxycarbonyl)-6-(trifluoromethyl)pyridin-2-yl]amino}cyclopentanecarboxylic acid and 1-phenylethanesulfonamide to give ethyl 5-cyano-6-{[3-({[(2-phenylethyl)sulfonyl]amino}carbonyl)cyclopentyl]amino}-2-(trifluoromethyl)nicotinate.

Yield: 68 mg (63%).

¹H NMR (600 MHz, DMSO-d₆): δ 1.25 (3H, t, J=7.2 Hz), 1.65-1.85 (4H, m), 1.87-1.94 (1H, m), 2.15-2.22 (1H, m), 2.71-2.78 (1H, m), 2.91-2.96 (2H, m), 3.62-3.68 (2H, m), 4.23 (2H, q, J=7.1 Hz), 4.32-4.40 (1H, m), 7.15-7.23 (3H, m), 7.23-7.29 (2H, m), 8.13-8.20 (1H, m), 8.41 (1H, s).

MS ^{m}/_{z}: 540 (M+1).

### Example 3

### Ethyl 6-{[3-({[(5-chloro-2-thienyl)sulfonyl]amino}carbonyl)cyclopentyl]amino}-5-cyano-2-(trifluoromethyl)nicotinate

Prepared according to Method A from ethyl 6-chloro-5-cyano-2-(trifluoromethyl)nicotinate and 5-chlorothiophene-2-sulfonamide to give ethyl 6-{[3-({[(5-chloro-2-thienyl)sulfonyl]amino}carbonyl)cyclopentyl]amino}-5-cyano-2-(trifluoromethyl)nicotinate. Yield: 87 mg (79%).

¹H NMR (600 MHz, DMSO-d₆): d 1.24 (3H, t, J=7.0 Hz), 1.60-1.84 (4H, m), 1.85-1.93 (1H, m), 2.13-2.21 (1H, m), 2.78 (1H, q, J=8.3 Hz), 4.23 (2H, q, J=7.1 Hz), 4.35 (1H, q, J=7.5 Hz), 7.23 (1H, d, J=4.1 Hz), 7.63 (1H, d, J=4.1 Hz), 8.12-8.18 (1H, m), 8.40 (1H, s).

MS ^{m}/_{z}: 550 (M−1).

### Example 4

### Ethyl 6-[(2-{[(5-chloro-2-thienyl)sulfonyl]amino}-2-oxoethyl)amino]-5-cyano-2-(trifluoromethyl)nicotinate

### (a) N-[3-Cyano-5-(ethoxycarbonyl)-6-(trifluoromethyl)pyridin-2-yl]glycine

TEA (0.5 mL, 6 mmol) was added to a solution of ethyl 6-chloro-5-cyano-2-(trifluoromethyl)nicotinate (341 mg, 1.2 mmol) and glycine (135 mg, 1.8 mmol) in EtOH (4.5 mL). The mixture was heated in a microwave reactor at 120° C. for 20 min. As starting material was still left more glycine (45 mg, 0.6 mmol) and TEA (0.3 mL) were added and the mixture was heated again in a microwave reactor at 120° C. for 20 min. Glycine was not completely dissolved. The mixture was evaporated, diluted with DCM and washed with 1% KHSO₄. The combined aqueous phases were extracted with DCM and the combined organic phases filtered through a phase separator and concentrated. The crude product was purified through prepHPLC [Kromasil C8, product loaded at low pH (0.2% HOAc in 5% CH₃CN) and after 10 min CH₃CN was gradually increased until 100% CH₃CN] to afford a white solid, N-[3-cyano-5-(ethoxycarbonyl)-6-(trifluoromethyl)pyridin-2-yl]glycine. Yield: 191 mg (50%)

¹H NMR (400 MHz, DMSO-d₆): δ 1.28 (3H, t, J=7.0 Hz), 4.04 (2H, d, J=6.4 Hz), 4.27 (2H, q, J=7.0 Hz), 12.71 (1H, s), 8.52 (1H, s).

MS m/z: 318 (M+1).

### (b) Ethyl 6-[(2-{[(5-chloro-2-thienyl)sulfonyl]amino}-2-oxoethyl)amino]-5-cyano-2-(trifluoromethyl)nicotinate

Prepared according to Method A from N-[3-cyano-5-(ethoxycarbonyl)-6-(trifluoromethyl)pyridin-2-yl]glycine and 5-chlorothiophene-2-sulfonamide to give ethyl 6-{[3-({[(5-chloro-2-thienyl)sulfonyl]amino}carbonyl)cyclopentyl]amino}-5-cyano-2-(trifluoromethyl)nicotinate. Yield: 87 mg (79%).

¹H NMR (600 MHz, DMSO-d₆): δ 1.24 (3H, t, J=6.9 Hz), 4.00-4.04 (2H, m), 4.23 (2H, q, J=6.9 Hz), 7.19 (1H, s), 7.56 (1H, s), 8.48 (1H, s).

MS ^{m}/_{z}: 550 (M−1).

### Example 5

### Ethyl 6-({2-[(benzylsulfonyl)amino]-2-oxoethyl}amino)-5-cyano-2-(trifluoromethyl)nicotinate

Prepared according to Method A from N-[3-cyano-5-(ethoxycarbonyl)-6-(trifluoromethyl)pyridin-2-yl]glycine and 1-phenylmethanesulfonamide to give ethyl 6-({2-[(benzylsulfonyl)amino]-2-oxoethyl}amino)-5-cyano-2-(trifluoromethyl)nicotinate. Yield: 44 mg (47%).

¹H NMR (600 MHz, DMSO-d₆): δ 1.26 (3H, t, J=7.1 Hz), 4.07-4.12 (2H, m), 4.25 (2H, q, J=7.0 Hz), 4.61 (2H, s), 7.25-7.30 (2H, m), 7.32-7.40 (3H, m), 8.54 (1H, s).

MS ^{M}/_{Z}: 469 (M−1).

### Example 6

### Ethyl 5-cyano-6-[(2-oxo-2-{[(2-phenylethyl)sulfonyl]amino}ethyl)amino]-2-(trifluoromethyl)nicotinate

Prepared according to Method A from N-[3-cyano-5-(ethoxycarbonyl)-6-(trifluoromethyl)pyridin-2-yl]glycine and 1-phenylethanesulfonamide to give ethyl 5-cyano-6-[(2-oxo-2-{[(2-phenylethyl)sulfonyl]amino}ethyl)amino]-2-(trifluoromethyl)nicotinate. Yield: 25 mg (26%).

¹H NMR (600 MHz, DMSO-d₆): δ 1.24 (3H, t, J=7.0 Hz), 2.90-2.95 (2H, m), 3.55-3.61 (2H, m), 4.07-4.12 (2H, m), 4.23 (2H, q, J=7.3 Hz), 7.15-7.21 (3H, m), 7.23-7.28 (2H, m), 8.48 (1H, s), 8.51 (1H, s).

MS ^{m}/_{z}: 483 (M−1).

### Example 7

### Ethyl 6-({3-[(benzylsulfonyl)carbamoyl]cyclopentyl}amino)-5-cyano-2-methylnicotinate

### (a) Ethyl 2-((dimethylamino)methylene)-3-oxobutanoate

Ethyl 3-oxobutanoate (250 mL, 1961 mmol) was stirred at r.t and 1,1-dimethoxy-N,N-dimethylmethanamine (327 mL, 2452 mmol) was added drop-wise. The reaction mixture was allowed to stir at r.t overnight. The reaction mixture was concentrated under vacuum and then azeotroped with toluene (3×300 mL) and placed under high vacuum to afford ethyl 2-((dimethylamino)methylene)-3-oxobutanoate as an oil, which was used without further purification. Yield: 363 g (100%).

MS ^{m}/_{Z}: 186 (M+1).

### (b) Ethyl 5-cyano-2-methyl-6-oxo-1,6-dihydropyridine-3-carboxylate

2-Cyanoacetamide (33.0 g, 392 mmol) was suspended in THF (250 mL) and slowly added to a suspension of NaH (60% dispersion in mineral oil, 16.5 g, 412 mmol) in THF (500 mL). The mixture was stirred for 2 h at r.t followed by the drop-wise addition of ethyl 2-((dimethylamino)methylene)-3-oxobutanoate (72.6 g, 392 mmol) suspended in THF (250 mL). The reaction mixture was stirred at r.t for 16 h and then acidified to pH 6 with acetic acid. Concentration under reduced pressure afforded crude material, which was suspended in 1 N HCl (1 L) and stirred for 30 minutes. The suspension was filtered and the product collected as a solid, which was azeotroped with Toluene (3×1 L) to afford ethyl 5-cyano-2-methyl-6-oxo-1,6-dihydropyridine-3-carboxylate as a solid. Yield: 75.3 g (93%).

¹H NMR (400 MHz, DMSO-d₆): δ 1.36 (3H, t, J=7.1 Hz), 2.62 (3H, s), 4.25 (2H, q, J=7.1 Hz), 8.71 (1H, s), 12.79 (1H, br s).

### (c) Ethyl 6-chloro-5-cyano-2-methylinicotinate

Ethyl 5-cyano-2-methyl-6-oxo-1,6-dihydropyridine-3-carboxylate (70.33 g, 341 mmol) was suspended in phosphoryl trichloride (124.5 mL, 1364 mmol) and the system heated at 100° C. overnight. The reaction mixture was cooled to r.t and concentrated under reduced pressure. The residue was diluted with DCM and poured onto ice. The biphasic mixture was stirred at r.t and slowly quenched with solid K₂CO₃ until all the POCl₃ had hydrolysed. The aqueous phase was extracted into DCM and the organics, dried (MgSO₄) and passed through a silica plug. The organics were concentrated under reduced pressure to afford ethyl 6-chloro-5-cyano-2-methylnicotinate as a solid, which was used without further purification. Yield: 61 g (80%).

¹H NMR (400 MHz, CDCl₃): δ 1.42 (3H, t, J=7.1 Hz), 2.91 (3H, s), 4.40 (2H, q, J=7.1 Hz), 8.49 (1H, s).

MS ^{m}/_{z}: 225 (+1).

### (d) 3-{[3-Cyano-5-(ethoxycarbonyl)-6-(methyl)pyridin-2-yl]amino}cyclopentanecarboxylic acid

DIPEA (1.0 mL, 5.7 mmol) was added to a solution of ethyl 6-chloro-5-cyano-2-methylnicotinate (748 mg, 3.3 mmol) and 3-aminocyclopentanecarboxylic acid (438 mg, 3.4 mmol) in EtOH (10 mL). The mixture was heated in a microwave reactor at 120° C. for min. As starting material was still left more 3-aminocyclopentanecarboxylic acid (119 mg, 0.9 mmol) was added and the mixture was heated in a microwave reactor at 120° C. for another 5 min. Saturated NH₄Cl(aq) was added and the mixture was extracted with DCM (3 times). The combined organic phase was filtered through a phase separator and evaporated. The crude product was purified by prepHPLC [Kromasil C8, Gradient 10 to 40% (0.1M NH₄OAc (aq) in 5% CH₃CN/CH₃CN)] to afford a white solid, 3-{[3-Cyano-5-(ethoxycarbonyl)-6-(methyl)pyridin-2-yl]amino}cyclopentanecarboxylic acid. Yield: 302 mg (29%)

MS ^{m}/_{z}: 318 (M+1).

### (e) Ethyl 6-({3-[(benzylsulfonyl)carbamoyl]cyclopentyl}amino)-5-cyano-2-methylnicotinate

DIPEA (0.2 mL, 1.1 mmol) was added to a solution of 3-{[3-Cyano-5-(ethoxycarbonyl)-6-(methyl)pyridin-2-yl]amino}cyclopentanecarboxylic acid (104 mg, 0.33 mmol) and TBTU (130 mg, 0.40 mmol) in dry DCM (5 mL) and the mixture was stirred for 20 min at r.t before 1-phenylmethanesulfonamide (74 mg, 0.43 mmol) was added and the reaction was left over night. Saturated NaHCO₃(aq) was added, the organic layer was separated and the aqueous phase was extracted with DCM. The combined organic phase was filtered through a phase separator and evaporated. The crude product obtained was purified by prepHPLC [Kromasil C8, Gradient 20 to 50% (0.1M NH₄OAc (aq) in 5% CH₃ CN/CH₃CN)] to give a white solid, ethyl 6-({3-[(benzylsulfonyl)carbamoyl]cyclopentyl}amino)-5-cyano-2-methylnicotinate. Yield: 97 mg (63%).

¹H NMR (500 MHz, DMSO-d₆): δ 1.30 (3H, t, J=7.1 Hz), 1.70-1.78 (1H, m), 1.79-1.88 (3H, m), 1.89-1.96 (1H, m), 2.14-2.21 (1H, m), 2.64 (3H, s), 2.73-2.80 (1H, m), 4.23 (2H, q, J=7.1 Hz), 4.49-4.57 (1H, m), 4.72 (2H, s), 7.29-7.32 (2H, m), 7.37-7.40 (3H, m), 7.70 (1H, d, J=7.3 Hz, NH), 8.28 (1H, s), 11.56 (1H, s).

MS ^{m}/_{z}: 471 (M+1).

1. A compound of formula I or a pharmaceutically acceptable salt thereof: 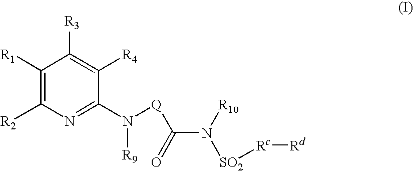
wherein
R₁ represents R₆OC(O), R₇C(O), R₁₆SC(O), R₁₇S, R₁₈C(S) or a group gII 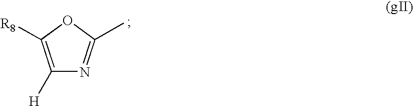
R₂ represents H, CN, halogen, NO₂, or (C₁-C₁₂)alkyl optionally interrupted by oxygen and/or optionally substituted by OH, aryl, cycloalkyl, heterocyclyl or one or more halogen atoms; further R₂ represents (C₁-C₁₂)alkoxy optionally substituted by one or more halogen atoms; further R₂ represents (C₃-C₆)cycloalkyl, hydroxy(C₁-C₁₂)alkyl, (C₁-C₁₂)alkylC(O), (C₁-C₁₂)alkylthioC(O), (C₁-C₁₂)alkylC(S), (C₁-C₁₂)alkoxyC(O), (C₃-C₆)cycloalkoxy, aryl, arylC(O), aryl(C₁-C₁₂)alkylC(O), heterocyclyl, heterocyclylC(O), heterocyclyl(C₁-C₁₂)alkylC(O), (C₁-C₁₂)alkylsulfinyl, (C₁-C₁₂)alkylsulfonyl, (C₁-C₁₂)alkylthio, (C₃-C₆)cycloalkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl, or (C₃-C₆)cycloalkyl(C₁-C₁₂)alkylsulfonyl or a group of formula NR^{a(2)}R^{b(2)} in which R^{a(2)} and R^{b(2)} independently represent H, (C₁-C₁₂)alkyl, or (C₁-C₁₂)alkylC(O) or R^{a(2)} and R^{b(2)} together with the nitrogen atom represent piperidine, pyrrolidine, azetidine or aziridine;
R₃ represents H, CN, NO₂, halogen, (C₁-C₁₂)alkyl optionally interrupted by oxygen and/or optionally substituted by OH, aryl, cycloalkyl, heterocyclyl or one or more halogen atoms; further R₃ represents (C₁-C₁₂)alkoxy optionally substituted by one or more halogen atoms; further R₃ represents (C₃-C₆)cycloalkyl, hydroxy(C₁-C₁₂)alkyl, (C₁-C₁₂)alkylC(O), (C₁-C₁₂)alkylthioC(O), (C₁-C₁₂)alkylC(S), (C₁-C₁₂)alkoxyC(O), (C₃-C₆)cycloalkoxy, aryl, arylC(O), aryl(C₁-C₁₂)alkylC(O), heterocyclyl, heterocyclylC(O), heterocyclyl(C₁-C₁₂)alkylC(O), (C₁-C₁₂)alkylsulfinyl, (C₁-C₁₂)alkylsulfonyl, (C₁-C₁₂)alkylthio, (C₃-C₆)cycloalkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl, or (C₃-C₆)cycloalkyl(C₁-C₁₂)alkylsulfonyl or a group of formula NR^{a(3)}R^{b(3)} in which R^{a(3)} and R^{b(3)} independently represent H, (C₁-C₁₂)alkyl, or (C₁-C₁₂)alkylC(O) or R^{a(3)} and R^{b(3)} together with the nitrogen atom represent piperidine, pyrrolidine, azetidine or aziridine;
R₄ represents H, CN, NO₂, halogen, (C₁-C₁₂)alkyl optionally interrupted by oxygen and/or optionally substituted by OH, COOH, (C₁-C₆)alkoxycarbonyl, aryl, cycloalkyl, heterocyclyl or one or more halogen atoms; further R₄ represents (C₃-C₆)cycloalkyl, hydroxy(C₁-C₁₂)alkyl, (C₁-C₁₂)alkylC(O), (C₁-C₁₂)alkylcycloalkyl, (C₁-C₁₂)alkoxy wherein the alkoxy group may optionally be substituted by one or more halogen atoms, OH and/or COOH and/or (C₁-C₆)alkoxycarbonyl; further R₄ represents (C₁-C₁₂)alkylthioC(O), (C₁-C₁₂)alkylC(S), (C₁-C₁₂)alkoxyC(O), (C₃-C₆)cycloalkoxy, aryl, arylC(O), aryl(C₁-C₁₂)alkylC(O), heterocyclyl, heterocyclylC(O), heterocyclyl(C₁-C₁₂)alkylC(O), (C₁-C₁₂)alkylsulfinyl, (C₁-C₁₂)alkylsulfonyl, (C₁-C₁₂)alkylthio, (C₃-C₆)cycloalkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl, or (C₃-C₆)cycloalkyl(C₁-C₁₂)alkylsulfonyl or a group of formula NR^{a(4)}R^{b(4)} in which R^{a(4)} and R^{b(4)} independently represent H, (C₁-C₁₂)alkyl, (C₁-C₁₂)alkylC(O) or R^{a(4)} and R^{b(4)} together with the nitrogen atom represent piperidine, pyrrolidine, azetidine or aziridine;
R₆ represents (C₁-C₁₂)alkyl optionally interrupted by oxygen, (with the proviso that any such oxygen must be at least 2 carbon atoms away from the ester-oxygen connecting the R₆ group) and/or optionally substituted by OH, aryl, cycloalkyl, heterocyclyl or one or more halogen atoms; further R₆ represents (C₃-C₆)cycloalkyl, hydroxy(C₂-C₁₂)alkyl, aryl or heterocyclyl;
R₇ represents (C₁-C₁₂)alkyl optionally interrupted by oxygen, and/or optionally substituted by OH, aryl, cycloalkyl, heterocyclyl or one or more halogen atoms; further R₇ represents (C₃-C₆)cycloalkyl, hydroxy(C₁-C₁₂)alkyl, aryl or heterocyclyl;
R₈ represents H, (C₁-C₁₂)alkyl optionally interrupted by oxygen, and/or optionally substituted by aryl, cycloalkyl, heterocyclyl or one or more halogen atoms; further R₈ represents (C₃-C₆)cycloalkyl, hydroxy(C₁-C₁₂)alkyl, (C₁-C₁₂)alkoxy, (C₃-C₆)cycloalkoxy, aryl, heterocyclyl, (C₁-C₁₂)alkylsulfinyl, (C₁-C₁₂)alkylsulfonyl, (C₁-C₁₂)alkylthio, (C₃-C₆)cycloalkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl or (C₃-C₆)cycloalkyl(C₁-C₁₂)alkylsulfonyl;
R₉ represents H or (C₁-C₁₂)alkyl;
R₁₀ represents H or (C₁-C₁₂)alkyl;
Q represents an unsubstituted or monosubstituted or polysubstituted (C₁-C₄)alkylene group, optionally interrupted by one or more groups/atoms selected among (C₃-C₇)cycloalkylene and a heteroatom being N, O or S, wherein any substituents each individually and independently are selected from (C₁-C₆)alkyl, (C₁-C₆)alkoxyl, oxy-(C₁-C₆)alkyl, (C₂-C₆)alkenyl, (C₂-C₆)alkynyl, (C₃-C₆)cycloalkyl, (C₃-C₆)cycloalkyl(C₁-C₄)alkylene, carboxyl, carboxy-(C₁-C₄)alkylene, aryl, aryl(C₁-C₄)alkylene, heterocyclyl, heterocyclyl(C₁-C₄)alkylene, nitro, cyano, halogeno, hydroxyl, and NR^{a(Q)}R^{b(Q)} in which R^{a(Q)} and R^{b(Q)} individually and independently from each other represents hydrogen, or (C₁-C₄)alkyl or R^{a(Q)} and R^{b(Q)} together with the nitrogen atom represent piperidine, pyrrolidine, azetidine or aziridine, with the proviso that any substituents are connected to Q in such a way that no quarternary ammonium compounds are formed (by these connections); further Q represents an unsubstituted or monosubstituted or polysubstituted (C₃-C₇)cycloalkylene wherein any substituents each individually and independently are selected from (C₁-C₆)alkyl, (C₁-C₆)alkoxyl, oxy-(C₁-C₆)alkyl, (C₂-C₆)alkenyl, (C₂-C₆)alkynyl, (C₃-C₆)cycloalkyl, (C₃-C₆)cycloalkyl(C₁-C₄)alkylene, carboxyl, carboxy-(C₁-C₄)alkylene, aryl, aryl(C₁-C₄)alkylene, heterocyclyl, heterocyclyl(C₁-C₄)alkylene, nitro, cyano, halogeno, hydroxyl, and NR^{a(Q)}R^{b(Q)} in which R^{a}(Q) and R^{b(Q)} individually and independently from each other represents hydrogen, (C₁-C₄)alkyl or R^{a(Q)} and R^{b(Q)} together with the nitrogen atom represent piperidine, pyrrolidine, azetidine or aziridine; further Q represents aryl wherein any substituents each individually and independently are selected from (C₁-C₆)alkyl, (C₁-C₆)alkoxyl, oxy-(C₁-C₆)alkyl, (C₂-C₆)alkenyl, (C₂-C₆)alkynyl, (C₃-C₆)cycloalkyl, (C₃-C₆)cycloalkyl(C₁-C₄)alkylene, carboxyl, carboxy-(C₁-C₄)alkylene, aryl, aryl(C₁-C₄)alkylene, heterocyclyl, heterocyclyl(C₁-C₄)alkylene, nitro, cyano, halogeno, hydroxyl, and NR^{a(Q)}R^{b(Q)} in which R^{a(Q)} and R^{b(Q)} individually and independently from each other represents hydrogen, (C₁-C₄)alkyl or R^{a(Q)} and R^{b(Q)} together with the nitrogen atom represent piperidine, pyrrolidine, azetidine or aziridine;
R₁₆ represents (C₁-C₁₂)alkyl optionally interrupted by oxygen and/or optionally substituted by OH, aryl, cycloalkyl, heterocyclyl or one or more halogen atoms; further R₁₆ represents (C₃-C₆)cycloalkyl, hydroxy(C₂-C₁₂)alkyl, (C₁-C₁₂)alkoxy, (C₃-C₆)cycloalkoxy, aryl or heterocyclyl;
R₁₇ represents (C₁-C₁₂)alkyl optionally interrupted by oxygen and/or optionally substituted by OH, aryl, cycloalkyl, heterocyclyl or one or more halogen atoms; further R₁₇ represents (C₃-C₆)cycloalkyl, hydroxy(C₁-C₁₂)alkyl, (C₁-C₁₂)alkoxy, (C₃-C₆)cycloalkoxy, aryl or heterocyclyl;
R₁₈ represents (C₁-C₁₂)alkyl optionally interrupted by oxygen and/or optionally substituted by OH, aryl, cycloalkyl, heterocyclyl or one or more halogen atoms; further R₁₈ represents (C₃-C₆)cycloalkyl, hydroxy(C₁-C₁₂)alkyl, (C₁-C₁₂)alkoxy, (C₃-C₆)cycloalkoxy, aryl or heterocyclyl;
R^{c} is absent or represents an unsubstituted or monosubstituted or polysubstituted (C₁-C₄)alkylene group, (C₁-C₄)oxoalkylene group, (C₁-C₄)alkyleneoxy or oxy-(C₁-C₄)alkylene group, wherein any substituents each individually and independently are selected from (C₁-C₄)alkyl, (C₁-C₄)alkoxyl, oxy-(C₁-C₄)alkyl, (C₂-C₄)alkenyl, (C₂-C₄)alkynyl, (C₃-C₆)cycloalkyl, carboxyl, carboxy-(C₁-C₄)alkyl, aryl, heterocyclyl, nitro, cyano, halogeno, hydroxyl, and NR^{a(Rc)}R^{b(Rc)} in which R^{a(Rc)} and R^{b(Rc)} individually and independently from each other represents hydrogen, (C₁-C₄)alkyl or R^{a(Rc)} and R^{b(Rc)} together with the nitrogen atom represent piperidine, pyrrolidine, azetidine or aziridine; further R^{c} represents imino (—NH—), N-substituted imino (—NR₁₉—), (C₁-C₄)alkyleneimino or N-substituted (C₁-C₄)alkyleneimino (—N(R₁₉)—((C₁-C₄)alkylene) wherein the mentioned alkylene groups are unsubstituted or monosubstituted or polysubstituted with any substituents according to above;
R₁₉ represents H or (C₁-C₄)alkyl; and
R^{d} represents (C₃-C₈)cycloalkyl, aryl or heterocyclyl, and anyone of these groups optionally substituted with one or more halogen atoms and/or one or more of the following groups, OH, CN, NO₂, (C₁-C₁₂)alkyl, (C₁-C₁₂)alkoxyC(O), (C₁-C₁₂)alkoxy, halogen substituted (C₁-C₁₂)alkyl, (C₃-C₆)cycloalkyl, aryl, heterocyclyl, (C₁-C₁₂)alkylsulfinyl, (C₁-C₁₂)alkylsulfonyl, (C₁-C₁₂)alkylthio, (C₃-C₆)cycloalkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl, or (C₃-C₆)cycloalkyl(C₁-C₁₂)alkylsulfonyl or a group of formula NR^{a(Rd)}R^{b(Rd)} in which R^{a(Rd)} and R^{b(Rd)} independently represent H, (C₁-C₁₂)alkyl, or (C₁-C₁₂)alkylC(O) or R^{a(Rd)} and R^{b(Rd)} together with the nitrogen atom represent piperidine, pyrrolidine, azetidine or aziridine.

2. A compound according to claim 1 wherein
R₂ represents H, CN, NO₂, or (C₁-C₆)alkyl optionally interrupted by oxygen and/or optionally substituted by OH, aryl, cycloalkyl, heterocyclyl or one or more halogen atoms; further R₂ represents (C₁-C₆)alkoxy optionally substituted by one or more halogen atoms; further R₂ represents (C₃-C₆)cycloalkyl, hydroxy(C₁-C₆)alkyl, (C₁-C₆)alkylC(O), (C₁-C₆)alkylthioC(O), (C₁-C₆)alkylC(S), (C₁-C₆)alkoxyC(O), (C₃-C₆)cycloalkoxy, aryl, arylC(O), aryl(C₁-C₆)alkylC(O), heterocyclyl, heterocyclylC(O), heterocyclyl(C₁-C₆)alkylC(O), (C₁-C₆)alkylsulfinyl, (C₁-C₆)alkylsulfonyl, (C₁-C₆)alkylthio, (C₃-C₆)cycloalkylthio, arylsulfinyl, arylsulfonyl, arylthio, aryl(C₁-C₆)alkylthio, aryl(C₁-C₆)alkylsulfinyl, aryl(C₁-C₆)alkylsulfonyl, heterocyclyl(C₁-C₆)alkylthio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkylsulfinyl, or (C₃-C₆)cycloalkyl(C₁-C₆)alkylsulfonyl or a group of formula NR^{a(2)}R^{b(2)} in which R^{a(2)} and R^{b(2)} independently represent H, (C₁-C₆)alkyl, or (C₁-C₆)alkylC(O) or R^{a(2)} and R^{b(2)} together with the nitrogen atom represent piperidine, pyrrolidine, azetidine or aziridine;
R₃ represents H, CN, NO₂, halogen, (C₁-C₆)alkyl optionally interrupted by oxygen and/or optionally substituted by OH, aryl, cycloalkyl, heterocyclyl or one or more halogen atoms; further R₃ represents (C₁-C₆)alkoxy optionally substituted by one or more halogen atoms; further R₃ represents (C₃-C₆)cycloalkyl, hydroxy(C₁-C₆)alkyl, (C₁-C₆)alkylC(O), (C₁-C₆)alkylthioC(O), (C₁-C₆)alkylC(S), (C₁-C₆)alkoxyC(O), (C₃-C₆)cycloalkoxy, aryl, arylC(O), aryl(C₁-C₆)alkylC(O), heterocyclyl, heterocyclylC(O), heterocyclyl(C₁-C₆)alkylC(O), (C₁-C₆)alkylsulfinyl, (C₁-C₆)alkylsulfonyl, (C₁-C₆)alkylthio, (C₃-C₆)cycloalkylthio, arylsulfinyl, arylsulfonyl, arylthio, aryl(C₁-C₆)alkylthio, aryl(C₁-C₆)alkylsulfinyl, aryl(C₁-C₆)alkylsulfonyl, heterocyclyl(C₁-C₆)alkylthio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkylsulfinyl, or (C₃-C₆)cycloalkyl(C₁-C₆)alkylsulfonyl or a group of formula NR^{a(3)}R^{b(3)} in which R^{a(3)} and R^{b(3)} independently represent H, (C₁-C₆)alkyl, or (C₁-C₆)alkylC(O) or R^{a(3)} and R^{b(3)} together with the nitrogen atom represent piperidine, pyrrolidine, azetidine or aziridine;
R₄ represents H, CN, NO₂, halogen, (C₁-C₆)alkyl optionally interrupted by oxygen and/or optionally substituted by OH, COOH, (C₁-C₆)alkoxycarbonyl, aryl, cycloalkyl, heterocyclyl or one or more halogen atoms; further R₄ represents (C₃-C₆)cycloalkyl, hydroxy(C₁-C₆)alkyl, (C₁-C₆)alkylC(O), (C₁-C₆)alkoxy wherein the alkoxy group may optionally be substituted by one or more halogen atoms, OH and/or COOH and/or (C₁-C₃)alkoxycarbonyl; further R₄ represents (C₁-C₆)alkylthioC(O), (C₁-C₆)alkylC(S), (C₁-C₆)alkoxyC(O), (C₃-C₆)cycloalkoxy, aryl, arylC(O), aryl(C₁-C₆)alkylC(O), heterocyclyl, heterocyclylC(O), heterocyclyl(C₁-C₆)alkylC(O), (C₁-C₆)alkylsulfinyl, (C₁-C₆)alkylsulfonyl, (C₁-C₆)alkylthio, (C₃-C₆)cycloalkylthio, arylsulfinyl, arylsulfonyl, arylthio, aryl(C₁-C₆)alkylthio, aryl(C₁-C₆)alkylsulfinyl, aryl(C₁-C₆)alkylsulfonyl, heterocyclyl(C₁-C₆)alkylthio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkylsulfinyl, or (C₃-C₆)cycloalkyl(C₁-C₆)alkylsulfonyl or a group of formula NR^{a(4)}R^{b(4)} in which R^{a(4)} and R^{b(4)} independently represent H, (C₁-C₆)alkyl, or (C₁-C₆)alkylC(O) or R^{a(4)} and R^{b(4)} together with the nitrogen atom represent piperidine, pyrrolidine, azetidine or aziridine;
R₆ represents (C₁-C₆)alkyl optionally interrupted by oxygen, (with the proviso that any such oxygen must be at least 1 carbon atom away from the ester-oxygen connecting the R₆ group) and/or optionally substituted by OH, aryl, cycloalkyl, heterocyclyl or one or more halogen atoms; further R₆ represents (C₃-C₆)cycloalkyl, hydroxy(C₂-C₆)alkyl, aryl or heterocyclyl;
R₇ represents (C₁-C₆)alkyl optionally interrupted by oxygen, and/or optionally substituted by OH, aryl, cycloalkyl, heterocyclyl or one or more halogen atoms; further R₇ represents (C₃-C₆)cycloalkyl, hydroxy(C₁-C₆)alkyl, aryl or heterocyclyl;
R₈ represents H, (C₁-C₆)alkyl optionally interrupted by oxygen, and/or optionally substituted by aryl, cycloalkyl, heterocyclyl or one or more halogen atoms; further
R₈ represents (C₃-C₆)cycloalkyl, hydroxy(C₁-C₆)alkyl, (C₁-C₆)alkoxy, (C₃-C₆)cycloalkoxy, aryl, heterocyclyl, (C₁-C₆)alkylsulfinyl, (C₁-C₆)alkylsulfonyl, (C₁-C₆)alkylthio, (C₃-C₆)cycloalkylthio, arylsulfinyl, arylsulfonyl, arylthio, aryl(C₁-C₆)alkylthio, aryl(C₁-C₆)alkylsulfinyl, aryl(C₁-C₆)alkylsulfonyl, heterocyclyl(C₁-C₆)alkylthio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkylsulfinyl or (C₃-C₆)cycloalkyl(C₁-C₆)alkylsulfonyl;
R₉ represents H or (C₁-C₆)alkyl;
R₁₀ represents H or (C₁-C₆)alkyl;
R₁₆ represents (C₁-C₆)alkyl optionally interrupted by oxygen and/or optionally substituted by OH, aryl, cycloalkyl, heterocyclyl or one or more halogen atoms; further R₁₆ represents (C₃-C₆)cycloalkyl, hydroxy(C₂-C₆)alkyl, (C₁-C₆)alkoxy, (C₃-C₆)cycloalkoxy, aryl, or heterocyclyl;
R₁₇ represents (C₁-C₆)alkyl optionally interrupted by oxygen and/or optionally substituted by OH, aryl, cycloalkyl, heterocyclyl or one or more halogen atoms; further R₁₇ represents (C₃-C₆)cycloalkyl, hydroxy(C₁-C₆)alkyl, (C₁-C₆)alkoxy, (C₃-C₆)cycloalkoxy, aryl or heterocyclyl;
R₁₈ represents (C₁-C₆)alkyl optionally interrupted by oxygen and/or optionally substituted by OH, aryl, cycloalkyl, heterocyclyl or one or more halogen atoms; further R₁₈ represents (C₃-C₆)cycloalkyl, hydroxy(C₁-C₆)alkyl, (C₁-C₆)alkoxy, (C₃-C₆)cycloalkoxy, aryl or heterocyclyl;
R^{d} represents (C₃-C₈)cycloalkyl, aryl or heterocyclyl, and anyone of these groups optionally substituted with one or more halogen atoms and/or one or more of the following groups, OH, CN, NO₂, (C₁-C₆)alkyl, (C₁-C₆)alkoxyC(O), (C₁-C₆)alkoxy, halogen substituted (C₁-C₆)alkyl, (C₃-C₆)cycloalkyl, aryl, heterocyclyl, (C₁-C₆)alkylsulfinyl, (C₁-C₆)alkylsulfonyl, (C₁-C₆)alkylthio, (C₃-C₆)cycloalkylthio, arylsulfinyl, arylsulfonyl, arylthio, aryl(C₁-C₆)alkylthio, aryl(C₁-C₆)alkylsulfinyl, aryl(C₁-C₆)alkylsulfonyl, heterocyclyl(C₁-C₆)alkylthio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkylsulfinyl, or (C₃-C₆)cycloalkyl(C₁-C₆)alkylsulfonyl or a group of formula NR^{a(Rd)}R^{b(Rd)} in which R^{a(Rd)} and R^{b(Rd)} independently represent H, (C₁-C₆)alkyl, or (C₁-C₆)alkylC(O) or R^{a(Rd)} and R^{b(Rd)} together with the nitrogen atom represent piperidine, pyrrolidine, azetidine or aziridine.

3. A compound according to claim 2 wherein:
R₁ represents R₆OC(O);
R₂ represents H, CN, NO₂, or (C₁-C₆)alkyl optionally interrupted by oxygen and/or optionally substituted by OH, aryl, cycloalkyl, heterocyclyl or one or more halogen atoms; further R₂ represents (C₁-C₆)alkoxy optionally substituted by one or more halogen atoms; further R₂ represents (C₃-C₆)cycloalkyl, hydroxy(C₁-C₆)alkyl, (C₁-C₆)alkylC(O), (C₁-C₆)alkylthioC(O), (C₁-C₆)alkylC(S), (C₁-C₆)alkoxyC(O), (C₃-C₆)cycloalkoxy, aryl, arylC(O), aryl(C₁-C₆)alkylC(O), heterocyclyl, heterocyclylC(O), or heterocyclyl(C₁-C₆)alkylC(O) or a group of formula NR^{a(2)}R^{b(2)} in which R^{a(2)} and R^{b(2)} independently represent H, (C₁-C₆)alkyl, or (C₁-C₆)alkylC(O) or R^{a(2)} and R^{b(2)} together with the nitrogen atom represent piperidine, pyrrolidine, azetidine or aziridine;
R₃ represents H, CN, NO₂, halogen, or (C₁-C₆)alkyl optionally interrupted by oxygen and/or optionally substituted by OH, aryl, cycloalkyl, heterocyclyl or one or more halogen atoms; further R₃ represents (C₁-C₆)alkoxy optionally substituted by one or more halogen atoms; further R₃ represents (C₃-C₆)cycloalkyl, hydroxy(C₁-C₆)alkyl, (C₁-C₆)alkylC(O), (C₁-C₆)alkylthioC(O), (C₁-C₆)alkylC(S), (C₁-C₆)alkoxyC(O), (C₃-C₆)cycloalkoxy, aryl, arylC(O), aryl(C₁-C₆)alkylC(O), heterocyclyl, heterocyclylC(O), heterocyclyl(C₁-C₆)alkylC(O), or (C₁-C₆)alkylsulfinyl, or a group of formula NR^{a(3)}R^{b(3)} in which R^{a(3)} and R^{b(3)} independently represent H, (C₁-C₆)alkyl, or (C₁-C₆)alkylC(O) or R^{a(3)} and R^{b(3)} together with the nitrogen atom represent piperidine, pyrrolidine, azetidine or aziridine;
R₄ represents H, CN, NO₂, halogen, or (C₁-C₆)alkyl optionally interrupted by oxygen and/or optionally substituted by OH, COOH, aryl, cycloalkyl, heterocyclyl or one or more halogen atoms; further R₄ represents (C₃-C₆)cycloalkyl, hydroxy(C₁-C₆)alkyl, (C₁-C₆)alkylC(O), (C₁-C₆)alkoxy wherein the alkoxy group may optionally be substituted by one or more halogen atoms, OH and/or COOH and/or methoxycarbonyl; further R₄ represents (C₁-C₆)alkylthioC(O), (C₁-C₆)alkylC(S), (C₁-C₆)alkoxyC(O), (C₃-C₆)cycloalkoxy, aryl, arylC(O), aryl(C₁-C₆)alkylC(O), heterocyclyl, heterocyclylC(O), or heterocyclyl(C₁-C₆)alkylC(O) or a group of formula NR^{a(4)}R^{b(4)} in which R^{a(4)} and R^{b(4)} independently represent H, (C₁-C₆)alkyl, or (C₁-C₆)alkylC(O) or R^{a(4)} and R^{b(4)} together with the nitrogen atom represent piperidine, pyrrolidine, azetidine or aziridine; and
R^{d} represents (C₃-C₈)cycloalkyl, aryl or heterocyclyl, and anyone of these groups optionally substituted with one or more halogen atoms and/or one or more of the following groups, CN, NO₂, (C₁-C₆)alkyl, (C₁-C₆)alkoxy, halosubstituted (C₁-C₆)alkyl, (C₃-C₆)cycloalkyl, aryl, heterocyclyl, (C₁-C₆)alkylsulfinyl, (C₁-C₆)alkylsulfonyl, (C₁-C₆)alkylthio, (C₃-C₆)cycloalkylthio, arylsulfinyl, arylsulfonyl, arylthio, aryl(C₁-C₆)alkylthio, aryl(C₁-C₆)alkylsulfinyl, aryl(C₁-C₆)alkylsulfonyl, heterocyclyl(C₁-C₆)alkylthio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkylsulfinyl or (C₃-C₆)cycloalkyl(C₁-C₆)alkylsulfonyl.

4. A compound according to claim 1 wherein:
R₁ represents R₆OC(O);
R₂ represents (C₁-C₆)alkyl optionally interrupted by oxygen and/or optionally substituted by one or more halogen atoms;
R₃ represents H;
R₄ represents CN or halogen;
R₆ represents (C₁-C₆)alkyl optionally interrupted by oxygen, (with the proviso that any such oxygen must be at least 2 carbon atoms away from the ester-oxygen connecting the R₆ group) and/or optionally substituted by OH, aryl, cycloalkyl, heterocyclyl or one or more halogen atoms;
R₉ represents H or (C₁-C₄)alkyl;
R₁₀ represents H or (C₁-C₄)alkyl;
Q represents an unsubstituted or monosubstituted or polysubstituted (C₁-C₄)alkylene group, wherein any substituents each individually and independently are selected from (C₁-C₆)alkyl, (C₁-C₆)alkoxyl, and oxy-(C₁-C₆)alkyl, or represents an unsubstituted or monosubstituted or polysubstituted (C₃-C₇)cycloalkylene wherein any substituents each individually and independently are selected from (C₁-C₄)alkyl, (C₁-C₄)alkoxyl, oxy-(C₁-C₄)alkyl and halogeno;
R^{c} is absent or represents an unsubstituted or monosubstituted (C₁-C₄)alkylene group, (C₁-C₄)alkyleneoxy or oxy-(C₁-C₄)alkylene group, wherein any substituents each individually and independently are selected from (C₁-C₄)alkyl; and
R^{d} represents aryl or heterocyclyl, and anyone of these groups optionally substituted with one or more halogen atoms and/or one or more of the following groups, CN, NO₂, (C₁-C₆)alkyl, (C₁-C₆)alkoxy, or halosubstituted (C₁-C₆)alkyl.

5. A compound according to claim 1 wherein:
R₁ is ethoxycarbonyl;
R₂ is methyl or trifluoromethyl;
R₃ is H;
R₄ is cyano;
R₆ is ethyl;
R₉ is H;
R₁₀ is H;
Q is a 1,3-cyclopentylene group or a methylene (—CH₂—) group;
R^{c} is absent or is methylene (—CH₂—) or ethylene (—CH₂CH₂—); and
R^{d} is phenyl or 5-chloro-2-thienyl.

6. A compound according to claim 1 which is of the formula (Ia):

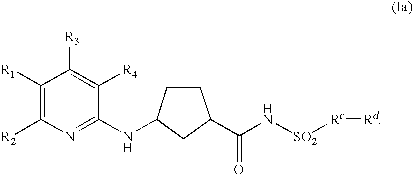

7. A compound according to claim 1 which is of the formula (Ib):

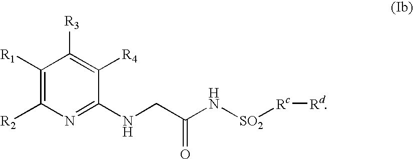

8. A compound according to claim 1 or claim 2 wherein R₁ represents R₆OC(O).

9. A compound according to claim 8 which is of the formula (Iaa):

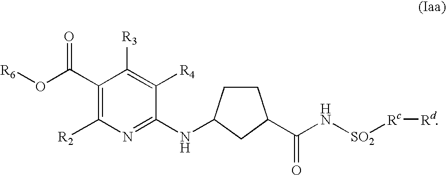

10. A compound according to claim 8 which is of the formula (Ibb):

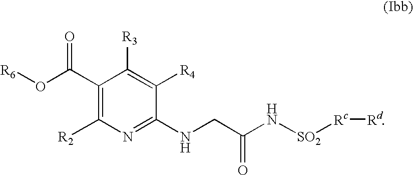

11. A compound according to claim 1 selected from:
ethyl 6-[(3-{[(benzylsulfonyl)amino]carbonyl}cyclopentyl)amino]-5-cyano-2-(trifluoromethyl)nicotinate
ethyl 5-cyano-6-{[3-({[(2-phenylethyl)sulfonyl]amino}carbonyl)cyclopentyl]amino}-2-(trifluoromethyl)nicotinate
ethyl 6-{[3-({[(5-chloro-2-thienyl)sulfonyl]amino}carbonyl)cyclopentyl]amino}-5-cyano-2-(trifluoromethyl)nicotinate ethyl 6-[(2-{[(5-chloro-2-thienyl)sulfonyl]amino}-2-oxoethyl)amino]-5-cyano-2-(trifluoromethyl)nicotinate
ethyl 6-({2-[(benzylsulfonyl)amino]-2-oxoethyl}amino)-5-cyano-2-(trifluoromethyl)nicotinate ethyl
6-({2-[(benzylsulfonyl)amino]-2-oxoethyl}amino)-5-cyano-2-(trifluoromethyl)nicotinate ethyl
6-({3-[(benzylsulfonyl)carbamoyl]cyclopentyl}amino)-5-cyano-2-methyl nicotinate;
and pharmaceutically acceptable salts thereof.

12. A pharmaceutical composition comprising a compound according to claim 1 and a pharmaceutically acceptable adjuvant, diluent and/or carrier.

13-15. (canceled)

16. A method of treatment of a platelet aggregation disorder comprising administering to a patient suffering from such a disorder a therapeutically effective amount of a compound according to claim 1.
